(19) United States
(12) Patent Application Publication
(10) Pub. No.: US 2009/0191608 A1
(43) Pub. Date: Jul. 30, 2009

(54) Pancreatic Islet Cell Preparation and Transplantation

(75) Inventor: Shinichi Matsumoto, Arlington, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 12/356,412

(22) Filed: Jan. 20, 2009

Related U.S. Application Data
(60) Provisional application No. 61/022,740, filed on Jan. 22, 2008.

(51) Int. Cl. C12N 9/48; C12N 5/06

(52) U.S. Cl. 435/212; 435/325

(57) ABSTRACT

The present invention includes compositions and methods for the preparation of pancreatic islet cells for transplantation.

### CROSS-REFERENCE TO RELATED APPLICATIONS

This application claims priority to U.S. Provisional Application Ser. No. 61/022,740, filed Jan. 22, 2008, the entire contents of which are incorporated herein by reference. INCORPORATION-BY-REFERENCE OF MATERIALS FILED ON COMPACT DISC None.

### TECHNICAL FIELD OF THE INVENTION

The present invention relates in general to the field of pancreatic islet transplantation, and more particularly, the new compositions and methods for improving the isolation, viability and transplantation of pancreatic islet cells.

### STATEMENT OF FEDERALLY FUNDED RESEARCH

None.

### BACKGROUND OF THE INVENTION

Without limiting the scope of the invention, its background is described in connection with islet cell transplantation.

Pancreatic Islet cell transplantation can be used to restore insulin production and glycemic control to the Type 1 (Juvenile) diabetic. Current results and toxicities do not justify wide-spread application, but improvements in both could yield to clinical (not experimental) application of this technology and could make this the preferred and leading treatment of Type 1 diabetes.

One such approach is found in U.S. Pat. No. 6,923,959, issued to Habener, et al. for a method of pre-inducing a state of immune tolerance before organ transplantation. Briefly, compositions and methods are described for the treatment of type I insulin-dependent diabetes mellitus and other conditions using newly identified stem cells that are capable of differentiation into a variety of pancreatic islet cells, including insulin-producing beta cells, as well as hepatocytes. Nestin has been identified as a molecular marker for pancreatic stem cells, while cytokeratin-19 serves as a marker for a distinct class of islet ductal cells. Methods are described in which nestin-positive stem cells can be isolated from pancreatic islets and cultured to obtain further stem cells or pseudo-islet like structures. Methods for ex vivo differentiation of the pancreatic stem cells are disclosed. Methods are described whereby pancreatic stem cells can be isolated, expanded, and transplanted into a patient in need thereof, either allogeneically, isogeneically or xenogenically, to provide replacement for lost or damaged insulin-secreting cells or other cells.

Another approach to increasing the viability of tissue for transplant is taught in U.S. Pat. No. 5,578,314, issued to Cochrum, et al., which discloses multiple layer alginate coatings of biological tissue for transplantation. Briefly, method for multiple layer coating of biological tissue and cells for transplantation is taught in which the cell or tissue transplants are coated with multiple coatings of purified alginate. The method includes applying the first coat of sodium alginate gelled with divalent cations followed by optional treatment with strontium, barium or other divalent cation, resuspending the single coated droplets in sodium alginate and forming the halo layer around the first coating via exchange or diffusion of divalent cations from the single coating to the surrounding soluble alginate, removing the excess coating and gelling the remaining thin layer of soluble alginate with divalent cations. The coated transplants have distinct structure where biological tissue or cell core is covered with the first alginate coat, which is surrounded by an intermediate halo layer which is covered by the outer coating.

United States Patent Application No. 20080009061, filed by Goto, et al., is directed to a method for preserving pancreatic islet, container for preserving pancreatic islet, and kit for transplanting pancreatic islet. Briefly, the method for preserving pancreatic islets includes a container for preserving pancreatic islet and a kit for transplanting pancreatic islet in order to effectively preserve the pancreatic islet.

United States Patent Application No. 20060189520, filed by Brand, et al., is directed to a treatment of diabetes with compositions and methods are provided for islet neogenesis therapy comprising a member of a group of factors that complement a gastrin/CCK receptor ligand, with formulations, devices and methods for sustained release delivery and for local delivery to target organs.

### SUMMARY OF THE INVENTION

The present invention was used to improve results in pancreatic islet allo-transplantation specifically by: (a) protecting against islet destruction from inflammation in the recipient by blocking interleukin-1 activation in the engraftment period with the administration of Anakinra (recombinant human interleukin-1 receptor antagonist, e.g., Kinert®), (b) enhance the yield of islets obtained from the donor pancreas by ductal injection of ET-Kyoto solution, and/or (c) enhancing the yield of islets obtained from the donor pancreas by the use of trypsin inhibition with trypsin inhibitor (e.g., ulinastatin) during pancreas digestion.

More particularly, the present invention includes compositions and methods of preparing a transplantable islet preparation, the method including, harvesting the pancreas of a donor; injecting the pancreatic ducts with ET-Kyoto solution or equivalent thereto; isolating pancreatic β-islet cells; and treating the patient with a human interleukin-1 antagonist at the time of islet transplant. In one embodiment, wherein the pancreatic β-islet cells are treated with a suitable collagenase, e.g., a human collagenase. In one specific example, the islets are processed in ET-Kyoto solution after their extraction from the pancreas. In one aspect, human interleukin-1 antagonist is selected from: one or more modifiers of interleukin-1 beta (IL-1β) gene transcription; one or more modifiers of IL-1β gene translation; one or more siRNAs that target the expression of IL-1β; one or more IL-1β receptors blockers; one or more interleukin-1 receptor antagonist proteins; one or more interleukin-1 receptor antagonist peptides; one or more active agents that modify the release of IL-1β; one or more antibodies that neutralize IL-1β; one or more antibodies that blocks an IL-1β receptor; one or more recombinant, naturally occurring IL-1β receptor antagonists; one or more anion transport inhibitors, lipoxins and alpha-tocopherol that inhibit the release of IL-1β; one or more opioids that inhibits a proteolytic enzyme that converts the inactive IL-1β precursor to its mature, active form; one or more antibodies that neutralizes the biological function of IL-1β, mixtures and combinations thereof. In one specific example, the IL-1β antagonist is anakinra. The method may further include concurrently providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.

Another aspect of the present invention is a method of preparing a transplantable islet preparation, the method including the steps of: harvesting the pancreas of a donor; injecting the pancreatic ducts with ET-Kyoto solution or equivalent thereto; isolating pancreatic β-islet cells from the harvested pancreas in the presence of a trypsin inhibitor; and treating the patient with a human interleukin-1 antagonist at the time of islet transplant. Examples of trypsin inhibitors include serum α-1 antitrypsin, a lima bean trypsin inhibitor, a Kunitz inhibitor, a ovomucoid inhibitor or a soybean inhibitor.

Yet another embodiment of the present invention is a method of preparing a transplantable islet preparation, by harvesting the pancreas of a donor; isolating pancreatic β-islet cells isolating pancreatic β-islet cells from the harvested pancreas in the presence of a trypsin inhibitor; and treating the patient with a human interleukin-1 antagonist and a Tumor Necrosis Factor antagonist at the time of islet transplant.

### BRIEF DESCRIPTION OF THE DRAWINGS

For a more complete understanding of the features and advantages of the present invention, reference is now made to the detailed description of the invention along with the accompanying figures and in which:

FIG. 1 shows the Islet yields before and after purification in the ductal injection group (DI) and the standard group (standard). Islet yields were significantly higher in DI group both before and after islet purification.

FIG. 2 shows the fasting blood glucose levels before and after islet transplantation of three patients in the DI group. All patients improved glycemic control after islet transplantation.

FIG. 3 shows that daily insulin doses before and after islet transplantation of three patients in the DI group. All three patients became insulin independent.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### LIST OF ABBREVIATIONS

| | |
|---|---|
| 2-D | Two-dimensional |
| ABO | Blood group system (A, B, AB and O) |
| AE | Adverse Event |
| AIDS | Acquired Immunodeficiency Syndrome |
| ALT | Alanine Aminotransferase |
| ANC | Absolute Neutrophyl Count |
| AST | Aspartate Aminotransferase |
| BCC | Basal Cell Carcinoma |
| BUMC | Baylor University Medical Center |
| BUN | Blood Urea Nitrogen |
| CBC | Complete Blood Count |
| CFR | Code of Federal Regulations |
| CMP | Complete Metabolic Panel |
| CRF | Case Report Form |
| DL | Deciliter |
| DM | Diabetes Mellitus |
| DRI | Diabetes Research Institute [Miami] |
| EBV | Epstein Barr Virus |
| EU | Endotoxin Units |
| FDA | Food and Drug Administration |
| Fr | French |
| G | Gauge |
| g | Gram |
| GFR | Glomerular Filtration Rate |
| Glofil | Iothalamate GFR assessment |
| Hb | Hemoglobin |
| HbA1c | Hemoglobin A1c |
| HBsAg | Hepatitis B surface antigen |
| HBV | Hepatitis B Virus |
| HCV | Hepatitis C Virus |
| Hg | Mercury |
| HIV | Human Immunodeficiency Virus |
| HLA | Human Leukocyte Antibody |
| HTLV- | Human T Lymphothrophic Virus I |
| HTN | Hypertension |
| ICT | Islet Cell Transplantation |
| ICU | Intensive Care Unit |
| IE | Islet Equivalents |
| IgG | Immunoglobulin G |
| IgM | Immunoglobulin M |
| IND | Investigational New Drug |
| INH | Isoniazide Hydrochloride |
| IRB | Institutional Review Board |
| IU | International Units |
| IV | Intravenous |
| IVGTT | Intravenous Glucose Tolerance Test |
| K | Potassium |
| kg | Kilogram |
| LDL | Low-Density Lipoproteins |
| LFT | Liver Function Tests |
| MAGE | Mean Amplitude of Glycemic |
| Mg | Magnesium |
| mg | Milligram |
| mL | Milliliter |
| mm | Millimeters |
| mm³ | Cubic Milliliter |
| MMF | Mycophenolate Mofetil (CellCept) |
| Na | Sodium |
| NCI | National Cancer Institute |
| ng | Nanogram |
| OGTT | Oral Glucose Tolerance Test |
| PA | Postero-anterior |
| PAK | Pancreas after Kidney Transplant |
| PCP | Pneumocystis carinii Pneumonia |
| PO | Per os or by mouth |
| PPD | Purified Protein Derivative |
| PRA | Panel Reactive Antibodies |
| PSA | Prostatic Specific Antigen |
| PT | Prothrombin Time |
| PTA | Pancreas Transplant Alone |
| PTT | Partial Thromboplastin Time |
| PV | Portal Vein |
| RBC | Red Blood Cells |
| SAE | Serious Adverse Event |
| SCC | Squamous Cell Carcinoma |
| SIR | Sirolimus |
| SPK | Simultaneous Pancreas-Kidney Transplant |
| TAC | Tacrolimus (Prograf) |
| TB | Tuberculosis |
| TC | Total Cholesterol |
| TGC | Triglycerides |
| UNOS | United Network for Organ |
| UW | University of Wisconsin |
| WBC | White Blood Cells |

Diabetes mellitus (DM) type 1 is a disease with significant social and economic impact. The prevalence of the disease in the United States is about 120,000 in individuals aged 19 or less and 300,000 to 500,000 at all ages and 150 million worldwide. There are 30,000 new cases diagnosed each year in the United States. DM is one of the most frequent chronic diseases in children in the United States¹. The cost of treatment and complications of this disease in the United States is 90 billion dollars a year.

The novel features of this invention include: (a) use of interleukin-1 blockade in the recipient of pancreatic islet cell transplants, (b) ductal preservation of the donor pancreas at the time of organ procurement by the preservative solution ET-Kyoto, and/or (c) the use of trypsin inhibition during donor pancreas digestion. ET-Kyoto solution, and the modifications thereto, inclue trehalose as a nonreducing disaccharide that stabilizes the cell membrane under various stressful conditions. Two variants on ET-Kyoto solution have different electrolyte contents, e.g., Na 100 mmol/L, K 44 mmol/L (so-called “extracellular” solution) and an “intracellular type” IT-Kyoto solution, e.g., Na 20 mmol/L, K 130 mmol/L, with trehalose at 35 gr/l. The complete solutions are summarized in Table 1.

TABLE 1
Preservation Solutions.

| | Solution | | | | | | | |
|---|---|---|---|---|---|---|---|---|
| | | | | | ET- | IT- | nEt- | |
| | E-C | C-S | UW | LPD-G | Kyoto | Kyoto | Kyoto | C |
| Na+ | 10 | 17 | 30 | 165 | 100 | 20 | 107 | 100 |
| K+ | 115 | 115 | 125 | 4 | 44 | 130 | 44 | 15 |
| Mg++ | 5 | 5 | 5 | 2 | — | — | — | 13 |
| Ca++ | — | — | — | — | — | — | — | 0.25 |
| Cl− | 15 | 15 | — | 101 | — | — | — | — |
| CO₃H− | 10 | 10 | — | — | — | — | — | — |
| PO₄H₂− | 58 | 58 | 25 | 36 | 26 | 25 | 25 | — |
| SO₄═ | 5 | 5 | 5 | — | — | — | — | — |
| Glucose | 195 | — | — | 56 | — | — | — | — |
| Gluconate | — | — | — | — | 100 | 100 | 100 | — |
| Lactobionate | — | — | 100 | — | — | — | — | 80 |
| Adenosine | — | — | 5 | — | — | — | — | 1 |
| Glutamine | — | — | 3 | — | — | — | — | 1 |
| Alopurinol | — | — | 1 | — | — | — | — | 1 |
| Trehalose | — | — | — | — | 120 | — | 120 | — |
| Raffinose | — | — | 30 | — | — | — | — | — |
| Dextran 40(g/L) | — | — | — | 20 | — | — | — | — |
| Mannitol(g/L) | — | 37.5 | — | — | — | — | — | 60 |
| EDTA(g/L) | — | 0.075 | — | — | — | — | — | — |
| HES(g/L) | — | — | 50 | — | 30 | 30 | 30 | — |
| NAC | — | — | — | — | — | — | 10 | — |
| Db c-AMP | — | — | — | — | — | — | 2 | — |
| Nitroglycerine | — | — | — | — | — | — | 0.44 | — |
| pH | 7.4 | 7.4 | 7.4 | 7.4 | 7.4 | 7.4 | 7.4 | 7.3 |
| Osmolarity(**) | 355 | 420 | 325 | 335 | 370 | 370 | 600 | 360 |

E-C: Euro-Collins. C-S: Collins-Sacks. UW: University of Wisconsin - Beltzer. LPD-G: Low potassium Dextran - Glucose. ET-K: Extracellular-type Kyoto. IT-K: Intracellular-type Kyoto. nET-K: new ET-K; C: Celsior. EDTA: ethylenediaminetetraacetic acid. HES: Hydroxyethyl starch. NAC: N-acetylcysteine. Db c-AMP: Dibutyl cyclic AMP. All concentración in mMol/L, except (*) gr/L.
(**)Osmolarity is expressed Osm/L.

Examples trypsin inhibitors include, but are not limited to, serum α-1 antitrypsin, a lima bean trypsin inhibitor, a Kunitz inhibitor, a ovomucoid inhibitor or a soybean inhibitor.

To date there are no mechanical devices able to effectively adjust the dose of insulin injected according to the serum glucose levels in patients with DM. This leads to less-than-perfect sugar control, with episodes of hypoglycemia which can be dangerous.

Pancreas Transplantation Benefits. Pancreas transplantation is a well-established treatment for type 1 DM. It is performed concomitantly with kidney transplantation [Simultaneous pancreas and kidney transplantation (SPK)], after kidney transplantation [“pancreas after kidney” (PAK)] or pancreas transplant alone (PTA). Simultaneous pancreas and kidney transplantation accounted for 75% of the pancreata transplanted in United States in 1999 and remains the procedure of choice for management for otherwise fit Type 1 diabetic patients under the age of 50 with renal failure². The indications for PTA, which make up less than 10% of the total numbers, are less objective but include life-threatening hypoglycemia unawareness necessitating continual presence of a caregiver and aggressive diabetic neuropathy. Relief of hypoglycemia unawareness is the most convincing reason to accept the risks of lifetime immunosuppression. It is this same group of patients selected for PTA who are also considered appropriate candidates for isolated islet cell transplantation.

The major achievements with pancreatic transplantation are insulin-independency and the avoidance, halting or regression of some of the complications related to DM. Life-style benefits from successful pancreas transplantation are unquestioned, and long-term normoglycemia can be achieved³⁻⁵. Perhaps the greatest benefit with respect to diabetic secondary complications is the improvement in autonomic and peripheral neuropathy; better cardiac function leads to better patient survival⁶. Not only is nerve conduction velocity improved, indicating neuronal repair within nerve sheaths, but also conduction amplitude is improved, indicative of axonal regeneration⁷. Transplantation must occur, however, before the onset of severe sensor motor neuropathy for the patient to derive the benefit. Usually, diabetic retinopathy does not improve post-transplant, as 90% of SPK patients already having permanent damage at time of transplantation 8.

Pancreas Transplantation Morbidity and Mortality. Pancreas transplantation is a well-established surgical procedure. It is considered a major surgical procedure associated with morbidity and mortality. Additional morbidity and mortality is related to the inherent immunosuppression therapy. The technique used requires en bloc transplantation of the whole pancreatic organ with both the exocrine and endocrine component together with the duodenal loop.

The specific complications related to the surgical procedure are vascular, anastomotic leaks, infectious and metabolic. These can result in mortality, repeat surgery and graft loss⁹. The most recent data suggests that technical failure rate is approximately 8% for SPK, 13% for PAK, and 11% for PTA. Graft thrombosis (typically venous) occurs in 2-14% of cases resulting in early graft loss¹⁰.

Specific complications are related to the type of intestinal drainage of the allograft: enteric or to the urinary bladder. With bladder drainage; complications include immediate postoperative hematuria, urinary leaks, urinary reflux pancreatitis, metabolic acidosis and dehydration from the secretion of fluid and bicarbonate by the exocrine pancreas into the bladder, and sterile cystitis due to the effect of the exocrine pancreatic enzymes on the bladder and urethral epithelium. In 8% to 23% cases, these complications necessitate surgical conversion to enteric drainage¹¹. With enteric drainage, the major complication is an anastomotic intestinal leak with intra-abdominal abscess formation, potentially leading to sepsis, multi-organ failure and death. A large number of complications mentioned above are related to the exocrine part of the transplanted pancreas or the transplanted duodenal loop. Despite the intense immunosuppression commonly used, the rejection rate after pancreas transplantation is around 30%, with 10% graft loss. Graft survival nationwide, as recorded by UNOS, is 88.5% at 3 months, 80% at one year, 52.9% at 3 years and 40.7% at 5 years. Results are better with kidney-pancreas transplants (87.7%, 83.8%, 77.2% and 67.5%, respectively). During a ten-year period (1991-2000), the annual death rate range was 36.3 to 82.3 per 1000 patients for pancreas transplants and 31.1 to 63.2 per 1000 patients with kidney-pancreas transplants¹².

Pancreatic Islet Cell Transplantation an Alternative to Whole Organ Pancreas Transplantation. The emerging alternative to whole organ pancreas transplantation is pancreatic islet cell transplantation (ICT). The process is based on the enzymatic isolation of the pancreatic islets of Langerhans from an organ procured from a cadaveric donor¹³⁻¹⁵; the islets obtained are injected into the liver of the recipient via percutaneous catheterization of the portal venous system¹⁶. This procedure allows the selective transplantation of the insulin-producing cell population avoiding open surgery as well as the transplantation of the duodenum and the exocrine pancreas and their related morbidity.

There are currently two trends in islet cell transplantation, using the immediate and delayed infusion approach. The immediate transplantation focuses on the use of the shortest time possible between islet isolation and islet infusion. An alternative method implies short-term culture of the islets after the isolation and before transplantation. This ensures increased purity of the islet isolate while it does not affect the viability and the function of the islets and seems to yield good results while the procedure is performed in a semi-elective setting^{17,18}.

Different anatomic locations were tried for the engrafting of the islet cells¹⁹⁻²¹. Currently, the portal vein is the preferred site of infusion, given the relative ease of access, the high venous flow with a double circulation system (arterial and portal venous) of the liver. The liver has a good regenerative capacity and is one of the major sites of insulin action. The liver site also seems to confer some immunological privilege to the islets. When compared to the whole organ pancreas transplant, the ICT has reduced surgical risk, is quicker and less expensive, is performed as an outpatient procedure and has therefore gained good patient acceptance.

The initial efforts with ICT had only modest results. The immunosuppression regimen was similar to the one used in solid organ transplantation, based on high dose steroids and calcineurin inhibitors—both agents with diabetogenic effects²². The results improved markedly with the changes in the manipulations of the islets^{13,15} and the change in immunosuppression, thus avoiding the higher doses of steroids and using sirolimus, tacrolimus and dacluzimab initiated by the investigators group at the University of Alberta in Edmonton, Canada. Their protocol requires, in general, two islet cell infusions to attain the critical cell mass necessary to achieve insulin-independency. The changes in treatment were adopted as the “Edmonton Protocol”, which is used in several transplant centers worldwide^{16, 23}. A recent report from the Edmonton group showed that 65 patients have received islet transplant at this center and 44 patients became insulin independent²⁴. At five year follow-up ˜80% showed presence of C-peptide indicating functioning transplanted islets, however, only ˜10% remained insulin free. Similar results have been reported from other centers within USA²⁵. In another recent advancement in this field, the Minnesota group have shown that marginal dose of islet cells isolated from a single donor pancreas are sufficient to achieve insulin independence in severely affected type I diabetic patients²⁶.

The morbidity related to the procedure includes complications related to the liver puncture, portal vein cannulation and elevation of the liver function tests (LFT). Complications related to the liver puncture are subcapsular or intra-parenchymal bleeding, intraperitoneal bleeding (cumulative frequency: 4% necessitating blood transfusion), gallbladder puncture (2%), biliary leaks (1%). Pneumothorax and/or hemothorax are exceedingly rare. Formation of fatty patches in the liver (steatosis) has been reported²⁷. It is likely that the incidence of these complications may be lowered with the use of smaller catheters and the use of ultrasonographic guidance to access the portal vein²⁴ and fibrin glue for closing hole of puncture in the liver. Complications of the portal vein cannulation and infusion include portal vein branch thrombosis (2%) and partial minor portal vein thrombosis (2%). In the series reported none of these necessitated surgery or another invasive procedure.

Transient elevation of the LFT is common (93% of cases,), as up to 46% of patients develop a significant rise (AST twice baseline or higher), but levels generally return to normal within two weeks of the transplant²⁸. Pain is encountered during the procedure, mainly due to the intercostal access and the rise in the portal pressure. Pain is uncommon after the procedure²⁹.

Donor factors include age, preexisting islet damage trauma, unrecognized DM, amyloid, fat infiltration, prolonged ICU stay, hemodynamic stability and inotropic medication requirements. The quality of the organ procurement is important, including avoidance of warm ischemia and pancreatic capsular injury.

The cold ischemia time (between donor cross-clamping and the start of the isolation) should not exceed 8 hours with regular transport media. This includes the transport and the storage of the donor pancreas while immersed in the University of Wisconsin (UW) solution. A novel approach to organ preservation uses the two-layer preservation technique³⁰. This involves the use of two solutions—University of Wisconsin (UW) solution and perfluorodecalin. Perfluorodecalin is a perfluorocarbon which has the ability to store oxygen and slowly deliver it to the organ stored, thus preserving the cellular ATP content, which is important for cell viability in the context of organ storage. The two-layered technique enables longer cold ischemia times, with equivalent results when comparing 6-8 hours of storage in the UW solution with up to 24 hours of storage with the two-layered method³⁰. Factors that influence isolation of clinical grade islets include: Optimal enzyme batch¹⁵, temperature control during the process, reagent quality, and islet culture. Previously we have shown that pancreatic duct preserved with M-Kyoto solution with ulinastatin³² improved pancreatic ducal integrity which is essential for collagenase delivery. With this technique clinical grade islets were successfully isolated from non-heart-beating donors³², therefore, we expect that we should be able to obtain transplantable islets from heart-beating donors in the present study.

Clinical grade islet recovery is achieved in 18-35% of the pancreata used. The islet cell infusion delivers 40-85% of the normal cell mass, but engraftment is estimated at 25-50%²⁹. Therefore, a second islet cell infusion is necessary in most cases in order to achieve insulin independence. The total number of pancreatic islets transplanted influences the achievement of insulin-independence. With the current isolation and preservation techniques infusion of a total of more than 9,000 islet-equivalents/kg is associated with a good graft outcomes; this is typically achieved with the use of two donor pancreata.

Recipient factors include anticoagulation and avoidance of cytokine activation and immunosuppression that avoids islet cell toxicity or insulin resistance.

The process of pancreatic islet isolation for transplant is performed in most centers in a specially designed facility in a clean environment using established protocols under the strict supervision of the FDA. The establishment of a new facility requires significant material investment followed by the appropriate validation process and necessitates skilled manpower³¹.

The focus of research in Islet Cell Transplants (ICT) is centered on the development of a safe and effective procedure that will eventually replace surgical pancreas transplantation together with an ideal immunosuppressive regimen that provides safe and effective prevention against rejection, while minimizing the side effects that negatively impact transplant recipient's quality of life.

Corticosteroids and high doses of calcineurin inhibitors as immunosuppressive agents have been associated with failure of the transplanted islets and return to insulin treatment. Using a regimen that provides adequate immunosuppression to prevent early and late rejection episodes, and minimizes steroid usage as well as high doses of calcineurin inhibitors as immunosuppressive agents is highly desirable.

This study is being conducted as a modification of the Edmonton protocol for ICT at our institution. Edmonton protocol is followed exception that: a) Etanercept and Anakinra may be administered during the early phase of the transplant to minimize the loss of islets due to inflammation which in turn will lead to improved islet engraftment; b) Thymoglobulin may be administered for induction instead of daclizumab; c) Sitaglipin (Januvia) may be used to enhance islet graft function. The use of Etanercept and Anakinra in this fashion is not described in the literature and to our knowledge is not currently applied in any islet cell protocol in this country. However the expected side effect toxicity is low and potentially considerable immunologic advantage can be gained from this approach: namely, being able to decrease Rapamycin or Tacrolimus doses if there is toxicity from these two agents. This use of Etanercept and Anakinra is one of the main ways our protocol is modified from Edmonton.

In addition, we will introduce new islet isolation protocol originally developed for non-heart-beating donor pancreas in Japan. Especially, pancreatic ductal preservation at the time of pancreas procurement, trypsin inhibition during pancreas digestion and islet friendly purification solutions should improve the quality and quantity of islets.

We are also enhancing the quality of life questionnaire (patient administered) with the goal of identifying factors which may improve patient compliance

Islet Culture. While some islet cell transplant centers still attempt to culture the cells prior to transplant for up to 72 hours, the consensus islet cell transplantation practice, including at Edmonton, still follows a “just-in time” pattern of transplanting the cells as soon as the isolation is complete and product release testing has been satisfactorily completed. The protocol of the present invention eliminates this as a requirement, and aims to perform transplantation as soon as possible after isolation and product release testing.

There may be instances, however, where culture of the cells is needed to allow for recipient preparation, or when an unforeseen event (for example positive crossmatch) forces us to use an alternative recipient for the cells prepared. In these situations culturing of the cells will prevent wasting the isolated cells. Given that the field in general is still debating the benefits to ‘cultured’ versus ‘fresh’ islets, any differences our study population has in time-to-transplant particularly as correlated to outcome should be noted.

Study design. An open-label, prospective single-center study was designed to assess the safety and efficacy of pancreatic islet-cell transplantation in patients with type 1 diabetes mellitus.

Study Duration. Patient participation will last for 2 years (24 months) post-final transplantation, and the enrollment period may be approximately 18 months. Patient enrollment is expected to be initiated in the second half of 2007. The study may be completed in 24 months after the last patient receives a final transplant.

Duration of Subjects Participation. Subject participation in the study may be for a period of 24 months after the final transplant. In addition, patients who are withdrawn from the study will continue to be followed for the entire 24 months duration of the study.

Study population: Sample Size. Patients included in this trial may be candidates for pancreatic islet cell transplant for type 1 diabetes mellitus. 15 patients may be enrolled in the study at a single center.

Recipient Inclusion and Exclusion Criteria. Eligibility for islet cell transplantation is determined by the Kidney and Pancreas Transplant Selection Committee at Baylor Regional Transplant Institute, similar to whole organ pancreas transplant candidates. Patients who are not eligible for whole organ pancreas transplantation will not be eligible for ICT. The process of evaluation for transplantation is performed prior to enrollment in the study.

Inclusion Criteria. Patient has been fully informed and has signed an IRB approved informed consent form and is willing and able to follow study procedures for the full 24 months.
1. Type 1 diabetes mellitus of more than 5 years duration
2. Age between 18 and 65
3. Unstable diabetes mellitus control despite expert management by a Diabetology care team for at least 6 months prior to consideration for transplantation as defined by the following:
   a) During the past six months (or during the period of intensive diabetes care): Any episodes of hypoglycemic unawareness, as defined by the inability to recognize glucose levels below 50 mg/dL; or episodes of loss of cognitive function; or frequent episodes of symptomatic hypoglycemia; or admission to the hospital for hypo- or hyperglycemia; and
   b) HbA1c>6.5
4. Psychogenically able to comply, in the opinion of the investigator
5. Female patients of childbearing potential must have a negative urine or serum pregnancy test upon hospitalization or within 7 days prior to enrollment and have agreed to utilize effective birth control throughout the study as well as for 6 weeks following study completion

Exclusion Criteria
1. Patient has previously received or is receiving an organ or bone marrow transplant.
2. Patient has a known hypersensitivity to Tacrolimus, Sirolimus, or CellCept®.
3. Patient is pregnant or lactating (must provide effective contraception method).
4. Patient has participated in a blinded trial or participated in a trial involving a non-marketed (investigational) drug within 3 months of enrollment.
5. Patient has participated in a trial involving a marketed drug or an infusion device within 30 days of the start of the trial.
6. Patient exhibits any one of the following clinical criteria:
   Glofl<60 mL/min
   Serum creatinine>1.6 mg/dL consistently
   Body mass index>28
   Malignancy other than BCC and SCC
   Radiographic evidence of pulmonary infection
   Evidence of liver disease as evidenced by >2×ULN for AST, ALT, Alk. Phos., or T. bili.
   Active infections
   Hypercoagulable states (history of recurrent venous thrombosis, defined thrombophilia)
   Bleeding/coagulation disorders
   Basal C-peptide >0.3 ng/mL
   HbA1c>12%
   Insulin requirement >1 IU/kg/day
   Seropositivity for HIV, HBV, HCV, HTLV-I
   Abnormal Pap smear, active gynecological infection
   Positive exercise or chemical tolerance test
   Patients currently under treatment for a medical condition requiring chronic use of steroids at a dose of prednisone >5 mg/day may be excluded.
   Substance/alcohol abuse
   Untreated proliferating diabetic retinopathy
   PPD conversion or positive PPD without INH
   No primary care physician or primary care physician less than 6 months
   Smoking in the last 6 months
   Abnormal CBC/Hemoglobin <12 g/dL
   Macroalbuminuria >300 mg/24 hrs
   Untreated hyperlipidemia—TC>200 mg/dL, TGC>200 mg/dL, LDL>130 mg/dL
   Untreated hyponatremia, hypokalemia, hypercalcemia, hypocalcemia
   Iodine contrast allergy
   PSA>4
   PRA>20%
   Active peptic ulcer disease/gallstones/hemangioma
   Abnormal mammogram.
   Patient receives any of the prohibited medications listed in section 6.8.

Pre-transplant Evaluation: Exams and Tests. All patients will undergo preliminary evaluation for acceptability. Following evaluation, patient files may be presented to the renal and pancreas transplant selection committee to determine their suitability for the islet transplant program.

### Physicians and Other Healthcare Professional Visits

Diabetologist
Transplant Nephrologist
Cardiologist
Transplant Surgeon
Social Worker
Transplant Nutritionist
Other consults are arranged according to clinical indication.

### Laboratory Tests

CBC, CMP, amylase, PT, PTT, INR, thyroid function tests (T4, TSH, FT4)
ABO blood type
PRA
HbA1c, C-peptide
Lipid panel
Urinalysis, urine drug screen, urine culture
HBsAg, HCV antibody, CMV IgM and IgG, EBV, HIV, HTLV-T, VZV IgG and IgM
Guaiac stool test
24 hour urinary microalbumin
Glofil
PSA (male patients after 45)
PPD

### Imaging and Other Tests

Chest X-rays, PA and lateral
EKG, 2-D echocardiogram and stress echocardiogram test
Doppler ultrasonogram of the liver
Lower extremity arterial Doppler study
Colonoscopy—patients after age 50 and/or with positive guaiac stool test
Mammogram (women after age 40), Pap smear
Eye exam by ophthalmologist to assess for eye problems related to diabetes such as diabetic retinopathy

Procedures. Organ Procurement and Transport. The procurement of the pancreas for islet isolation is performed from a cadaveric donor as part of standard organ procurement according to the United Network for Organ Sharing (UNOS) guidelines in place nationwide. The organ procurement is performed by a qualified transplant surgery group in conjunction with a local Organ Procurement Organization (OPO). The surgeons and OPO must be familiar with harvesting and shipping pancreata for islet cell isolation. In addition, they must have the proper equipment and shipping materials for longer cold ischemia times.

The donor pancreas is shipped to the processing facility according to UNOS regulations for the standard donor pancreas. It is stored during the transport in University of Wisconsin (UW) solution alone or with oxygenated perfluorocarbon (PFC) solution or an appropriate shipping medium. Pancreatic duct is also preserved with M-Kyoto solution with ulinastatin32 or an appropriate preservation solution.

Every effort may be made to transplant the islet cells as soon as they are deemed ready by the laboratory team and the Medical Director in each and every instance. Study subjects will not be assigned different timelines for each of the steps of this study (procurement, isolation, recipient preparation, islet infusion). However there are likely to be logistical delays at the donor operation, or in the laboratory work to separate the islets, or in the scheduling of the radiology suite, or in the preparation of the recipient. To prevent wastage of the cells, storage before isolation may be extended with the addition of perfluorocarbon to the University of Wisconsin solution, and storage after isolation but before transplantation may be extended with culture of the islets in an incubator. Because these timelines may vary somewhat from patient to patient, the differences in the time points between patients may be noted and correlated to success or failure to establish glycemic control. Likewise the use of perfluorocarbon solution, and/or the use of culture of the islets may be correlated between patients.

Donor Selection Criteria for Islet Cell Transplantation: Islet Cell Transplant—Donor Specific Inclusion Criteria: Multi-organ donor; Adequate in situ hypothermic perfusion; Maximum 18 hour cold ischemia in the above conditions; Minimum 15 years to 70 years old.

Islet Cell Transplant—Donor Specific Exclusion Criteria
a) Pre-existing diseases:
   Diabetes mellitus type 1 or 2
   Malignancies other than primary brain tumor
   Septicemia

General Donor Preclusions/Exclusions. The following are frequently encountered disease states or other conditions, which may be absolute grounds for rejection of a potential donor. In addition, a potential donor may be excluded for any reason if deemed necessary by the investigator
a) Clinical or active viral Hepatitis (A, B, or C)
b) Acquired Immunodeficiency Syndrome (AIDS)
c) HIV seropositivity (HIV-I or HIV-II)
d) HTLV-I or II
e) Syphilis
f) Active viral encephalitis or encephalitis of unknown origin
g) Creutzfeldt-Jacob Disease
h) Rabies
i) Treated or Active Tuberculosis
j) Septicemia
k) Dementia
l) Individuals who have received pit-hGH (pituitary growth hormone)
m) Malignancies except primary brain tumors
n) Serious illness of unknown etiology

Donor Behavior—History Exclusionary Criteria:
a) Men who have had sex with another man in the past five years
b) Persons who have reported non-medical intravenous, intramuscular, or subcutaneous injection of drugs in the past five years
c) Persons with hemophilia or related clotting disorders who have received human derived clotting factor concentrates
d) Men and women who have engaged in prostitution in the last five years
e) Sexual partners of persons described above
f) Persons who have been exposed in the preceding 12 months to known or suspected HIV infected blood through accidental needle stick or through contact with an open wound, non-intact skin, or mucous membrane
g) Persons who have received a tattoo, ear and/or body piercing, or acupuncture within 12 months preceding tissue donation
h) Inmates of correctional systems.

Laboratory and Other Medical Exclusionary Criteria of the Donor
a) Persons who cannot be tested for HIV infection because of refusal, inadequate blood samples (e.g. hemodilution that could result in false-negative tests), or any other reasons
b) Persons with a repeatedly reactive screening assay for HIV-I or HIV-II antibody regardless of the results of supplemental assays.
c) Persons whose history, physical examination, medical records, or autopsy reports reveal other evidence of HIV infection or high-risk behavior, such as a diagnosis of AIDS, unexplained weight loss, night sweats, blue or purple spots on the skin or mucous membranes typical of Kaposi's sarcoma, unexplained lymphadenopathy lasting >1 month, unexplained temperature >100.5° F. (38.6° C.) for >10 days, unexplained persistent cough and shortness of breath, opportunistic infections, unexplained persistent diarrhea, male-to-male sexual contact, sexually transmitted diseases, or needle tracks or other signs of parenteral drug abuse.

Pancreatic Islet Isolation. Isolation of the islets from donor pancreata will occur in the Baylor University Medical Center Islet Cell Processing Laboratory (ICPL) using modified the “automated method” described by Ricordi, et al.¹⁵ The ICPL includes a Class 10,000 clean suite for processing islets, a QA/QC laboratory to perform product release testing and a freezer room to store samples and reagents. The ICPL has so far performed twenty nine islet isolations for validation. Furthermore, the laboratory has processed five islet products for transplants under a FDA approved protocol 11731A to test the safety and efficacy of remote site isolated islet products. The remote site validation protocol is simultaneously conducted in collaboration with the Diabetes Research Institute in Miami, Fla. Recently ICPL performed 8 islet isolations with clinical grade pancreata and five isolated islets were successfully transplanted into four type 1 diabetic patients. More recently we performed three additional islet isolations for validation using collagenase enzyme from SERVA. Islet yield and the quality of all three isolations would have qualified for transplantation according to this protocol.

Human cadaveric donor pancreas may be received into the ICPL and islets may be isolated according to methods previously validated by the laboratory. All manipulations of the organ, islets and islet cell products are performed in Class 100 BioSafety cabinets which are contained in the class 10,000 clean suite.

These methods are as follows: Pancreas is acquired through an organ procurement organization (OPO) and shipped in Transport media. Preferably pancreatic duct is also preserved with M-Kyoto solution with Ulinastatin or an adequate preservation solution. The media will vary depending upon which OPO procures the organ. This varying media/transport may be carefully studied.
a) The organ is then transported to the BUMC ICPL. The cold ischemia time may be recorded and will vary depending upon the organ procurement method.
b) Trained personnel of laboratory receive the pancreas into a class 10,000 clean room and aseptically remove the organ from the transport media.
c) The pancreas is cleaned in a class 100 BioSafety cabinet in class 10,000 clean suite, if necessary. Cleaning consists of the removal of fat and non-endocrine tissue. After the cleaning is completed, the organ is dipped into a series of solutions to prevent the spread of any potential contaminant from procurement process. The cleaned organ is placed into a solution of betadine followed by dipping into an antibiotic solution containing a mixture of gentamycin, amphotericin B, and cefazolin. Finally the organ is placed in sterile Hank's buffer.
d) The cleaned pancreas is then perfused with a sterile collagenase enzyme which initiates the digestion of the pancreatic tissue.
e) Once perfusion is complete, the pancreas is cut into smaller pieces. The pieces along with the enzyme solution are placed into sterile “Ricordi chamber” connected in circuit with a heating coil and a collection reservoir¹⁵. The chamber contains sterile marbles which are used for mechanical disruption of the pancreatic tissue. The chamber is manually shaken slowly and the temperature of the enzyme solution is increased to 37° C. to liberate the islets and the pancreatic digest is periodically monitored for the appearance of “free” islets.
f) At an appropriate time the enzymatic action is arrested by diluting the pancreatic digest with cold buffer and treatment with human serum albumin. The digested tissue is collected into sterile disposable Erlenmeyer flasks.
g) The islets are separated from the acinar tissue using gradient centrifugation on a COBE2991 blood cell processor.
h) The purified islets are then placed in a serum-free CMRL 1066 based culture media containing human serum albumin.
i) The islets are transplanted immediately or are cultured for up to 72 hours in an atmosphere of 5% CO₂.

Validation Procedures—Release Testing Before Islet Infusion. Testing for each islet preparation final product includes islet cell counts, purity, viability, sterility, endotoxin and potency. The results of islet cell counts, purity, viability and endotoxin, are available prior to infusion, and must meet assay lot release criteria. The final results of the sterility and potency tests are not available until after infusion. If these results do not meet release criteria, corrective steps are taken as soon as the results are known. In addition, the product of islet isolation is tested prior to determining final disposition. If the interim tests do not pass release criteria, the cells will not be transplanted.

Criteria for release of islet preparation for infusion.
ABO compatibility between donor and recipient and negative cross match
Islet mass≧4000 IE/kg unless additional infusions are determined necessary by the Medical Director
Negative Gram stain and cultures up to the day of transplant
Endotoxin load≦5 EU/kg recipient body weight
Viability≧70%
Purity≧30%

Post-Transplant Testing
Glucose stimulated insulin release testing is performed after transplant and the Insulin Release Stimulation index should be greater than 1.
The final results of sterility cultures are available only after transplantation and should be negative.

Islet Cell Infusion: Location. The islet cell infusion is performed in the Interventional Radiology Suite at Baylor University Medical Center or Baylor All Saints Medical Center by an interventional radiologist. The procedure takes place in a suite designed for invasive procedures using sterile technique with access to general anesthesia if necessary.

Preparation and Anesthesia. The patient is admitted and prepared for the procedure. Informed consents are obtained for the procedure.

The lower right lateral chest the upper right abdomen and the epigastric area are prepped sterile with iodine-based preparation. Local anesthesia with IV sedation usually suffices. Local anesthesia is performed using the anesthetic of choice as determined by the Interventional Radiologist, with intercostal nerve block of the area.

Cannulation of the portal vein. Guidance, for the portal vein cannulation is obtained with real-time ultrasonography using a 3.5 MHz probe.

Puncture site. The procedure is performed by percutaneous direct puncture of the liver. The right or the left branch of the portal vein can be chosen for cannulation and the puncture site is chosen accordingly by the interventional radiologist.

Technique. A 22 G Chiba needle is used for access to the portal vein, following by the catheterization of the portal vein over a guide wire using the Seldinger technique. A 4-5 Fr catheter is introduced in the portal vein. Needle and catheter size may change at the discretion of the interventional radiologist performing the procedure.

Portogram. A portal venogram is obtained through the catheter, with manual injection of low osmolar iodinated contrast, in order to evaluate anatomy and flow. Minimal contrast use is recommended.

Islet Cell Infusion, The Bag System. The islet cell infusion bag system is composed of a 600 mL infusion bag containing the islet suspension with a volume of 200 mL. The infusion of islet cells uses 1 or 2 bag systems. More than one bag is needed when the islet volume for infusion exceeds 5 mL. Each bag containing islets has 35 IU/kg heparin added. The maximum dose of heparin in the infusion is 70 IU/kg. If the infusion is terminated prematurely, the remainder of the heparin dose should be calculated to reach a total of 35 IU/kg and should be given into the portal vein followed by a normal saline flush.

The content of the bag is infused using gravitation only into the portal venous system of the recipient. The bag is then flushed with 50 mL of Transplant Media and the flush is infused from the bag into the portal system. The procedure is then repeated with the other bag or bags containing islets.

Completion of the Infusion. After the infusion is completed, the infusion catheter and the bag are rinsed with an additional transplant media, making sure that no islets are trapped in bag ports or 3-way stopcock. The portal venogram is not repeated after the infusion to avoid islet toxicity.

Portal Venous Pressure Assessment. The portal venous pressure is obtained by direct measurement inline via 3-way connector. Measures are read on a cardiovascular monitor after appropriate zeroing of the system.

Timing of Portal Vein Measurement. Portal vein (PV) pressures may be obtained before the procedure, halfway during each islet cell bag infusion and at the end of each wash of the bag with rinse solution. The final portal pressure is documented as well.

Management of Changes in Portal Venous Pressures. The portal venous pressure is expected to rise during the islet cell infusion. The following situations require adjustment of the treatment: Portal vein pressure above 20 mm Hg before the procedure is a contraindication for islet cell infusion.

If at any time during the infusion the PV pressure exceeds twice the baseline value but is less than 18 mm Hg, the infusion may be held for 10 minutes and the pressure may be measured again. If the pressure is below twice the baseline and less than 18 mm Hg the infusion may be resumed. If not, another measurement is made 10 minutes later.

If the PV pressure exceeds twice the baseline but is below 18 mm Hg the procedure may continue. If at any time the PV pressure exceeds 22 mm Hg, the infusion is held until the pressure falls below 18 mm Hg. If the PV pressure is above 22 mm Hg longer than 10 minutes, or above 18 mm HG more than 20 minutes, the procedure is terminated.

Removal of the Portal Vein Catheter. The portal vein catheter is removed and the introducer sheath is then withdrawn until the tip is in the parenchyma. A hemostatic agent of the Radiologist's choice is placed in the tip of an iodine filled syringe and injected into external end of sheath. The hemostatic agent is further advanced to internal end of sheath using a stiffener/trocar/wire as chosen by the radiologist. The sheath is then withdrawn over the plug. The plug should be easily visualized within the liver parenchyma at this point. A second plug is placed if possible.

Recovery. Following the procedure the patient is observed in the Interventional Radiology recovery area for as long as necessary as determined by a Physician and then transferred to the Transplant Service for an overnight stay. Liver function tests and a Doppler ultrasonogram of the liver are obtained the day after the procedure.

Hospital stay. After recovery, the patient is admitted to the hospital on the Transplant Service for a 1-2 day observation. Length of stay may be determined by how the patient tolerates the initial dose of Thymoglobulin on Day 0. Patients will return to the hospital to receive subsequent dosing of Thymoglobulin on Day 2, 4 and 6 post-transplant. Criteria for discharge from hospital include: Laboratory test results which are not indicative of bleeding, including; but not exclusively; hemoglobin and hematocrit levels. LFT's within acceptable limits (less than twice upper limit of normal), and patent main, left and right PV with no significant bleed or collection per Doppler ultrasonogram performed the day after the islet cell infusion.

Repeat Islet Cell Infusion. The interim result of the first islet cell transplant may be assessed, in accordance with the scheduled procedures (see section 7). A second infusion is likely necessary in most patients but not mandatory. All steps associated with the first procedure (5.1 to 5.7) should be repeated with the subsequent infusions. Patients will return to day-I for the second, and if necessary, third infusion, Dosing will start with day 7 and follow the procedures as written. The need for subsequent islet cell infusions is not considered failure of treatment. Patients may receive a total of three islet infusions, if necessary.

The following criteria may be used for deciding when to proceed with subsequent transplants: (1) when the patient received less than the optimal dose of >10,000 Islet Equivalents/kg body weight and/or has not achieved insulin independence based on previous transplant(s); and (2) the patient does not have any unresolved serious adverse event(s) related to previous transplant or immunosuppression.

Study Medication. Insulin and Glycemic Control. Insulin dose may be gradually decreased as islet function improves and may be discontinued when the recipient achieves good glucose control (serum glucose range: 80-120 mg/dL) with HbA1C below 7% and with positive C-peptide levels.

Glucose Monitoring. The patient will receive a LifeScan OneTouch Ultra capillary blood glucose meter, an FDA-approved measuring device, which displays the real-time glucose measurement to the patient, connected to GLUCOMON™, an investigational communication device, which communicates wirelessly to a computer operated by the Principal Investigator or his designee.

Determination of the mean amplitude of glycemic excursions (MAGE), an index of glycemic lability, may be performed using eight capillary glucose meter readings a day for two consecutive days. The tests may be performed fasting, 2 hours after a meal, at 10 PM and 3 AM (optional). The MAGE may be determined pre-transplant (see pre-transplant evaluation), at day 21 after the first islet cell transplant and monthly post-transplant.

Subjects will undergo an intravenous glucose tolerance test (IVGTT). The IVGTT may be performed at Day 28 and Month 6 post-transplant. After an overnight fast, an intravenous line is inserted. A baseline sample is drawn via phlebotomy for glucose and C-peptide levels and then 50% dextrose (300 mg/kg) is given intravenously over 1 minute. Samples are obtained via fingerstick over the next 30 minutes for, glucose determinations at 0, 3, 5, 10, 20 and 30 min, with 0 time being defined as the beginning of the infusion. Samples are also drawn via phlebotomy for glucose and C-peptide 30 minutes post-infusion.

Hemoglobin Alc. Medication for Glycemic Control. Sitaglipin (JANUVIA®) may be administered orally starting immediately post-transplant 100 mg once per day and subjects will continue medication indefinitely. Dosage may be adjusted by physician based on glycemic control and medication side effects.

Intensive insulin therapy for the first month after islet transplantation. Intensive insulin therapy after islet transplantation will improve the engraftment of transplanted islets. For this purpose we will continue intensive insulin therapy at least one month. As default the candidate for islet transplantation uses the intensive insulin therapy for managing their diabetes.

The intensive insulin therapy is defined as more than 3 times blood glucose measurement per day followed by more than 3 times insulin injection (subcutaneous) per day or insulin pump use for continuous insulin injection. For multiple insulin injections, long-acting insulin (ex. Glargine/Levemir) and rapid-acting insulin (ex. Lispro/Aspart) are typically used. Injection times are typically before dinner or sleep for long-acting Insulin and before every meal for rapid-acting insulin. When patients use a pump, basal insulin is equivalent to long-acting insulin and bolus insulin is equivalent to short-acting insulin. (Patients can use only one type of insulin in a pump). If they take boluses of another type of insulin, those doses would have to be administered subcutaneously.

After islet transplantation, oral food intake may be held for a minimum of 8 hours and intravenous insulin therapy may be used. When the oral food intake starts, the patient will resume intensive insulin therapy. The amount of insulin may be decreased as needed.

POD 0: Intravenous insulin therapy may be administered as per current Baylor Health Care System standard protocol.

Immunosuppression: Etanercept (ENBREL®). May be administered intravenously at starting dose of 50 mg within the immediate pre-transplant period. Subsequent doses may be given subcutaneously at a dose of 25 mg on days 3, 7 and 10 post transplant.

Tacrolimus (PROGRAF®). May be initiated orally—starting dose of 1 mg PO every 12 hours starting within the immediate pre-transplant period. The administered dose may be modified so to achieve a whole blood trough concentration of 3-6 ng/mL within 72 hours of initial dose and maintain this range.

Sirolimus (RAPAMUNE®). May be initiated at a loading dose of 0.2 mg/kg PO single dose before transplant. The dose may be then lowered to 0.1 mg/kg/day PO and adjusted so to maintain a drug concentration level of 12-15 ng/mL during the first three months of treatment. After three months of treatment, the dose may be adjusted so to maintain a drug concentration level of 7-10 ng/mL.

Anti-thymocyte Globulin (Rabbit) (THYMOGLOBULIN®) May be administered intravenously—starting dose of 1.5 mg/kg body weight given in the peri-transplant period. Subsequent doses may be given intravenously at a dose of 1.5 mg/kg on days 2, 4 and 6 post-transplant. Premedications include the administration of up to 500 mg of intravenous methylprednisolone, 650 mg of acetaminophen and 25-50 mg of diphenhydramine. Since the administration of thymoglobulin could result in thrombocytopenia and leukopenia, the calculated thymoglobulin dose may be reduced by 50% if the platelet count is between 50,000 and 100,000 cells/mm³ or if the white blood cell count is between 2000 and 3000 cells/mm. The thymoglobulin dose may be held if the platelet count is less than 50 000 cells/mm³ or if the white blood cell count is less than 2000 cells/mm³.

Mycophenolate Mofetil (CELLCEPT®). May be used as an alternative to other medications if toxicity is present: administer 2-3 gm/day PO with initial dose (divided into 2 equal doses) for the initial dose and for the duration of the study. May be administered via IV or capsule formulation. Dose may be changed due to adverse events.

Mycophenolic acid delayed-release tablet (Myfortic). May be used as an alternative to other medications if toxicity is present: administer 1440 mg/day PO with initial dose (divided into 2 equal doses) for the initial dose and for the duration of the study. Dose may be changed due to adverse events.

Anakinra (KINERET®). May be administered subcutaneously—starting dose of 100 mg given within the immediate pre-transplant period. Subsequent doses may be given subcutaneously at a dose of 100 mg on days 1 to 7 POD.

Anticoagulation. The islet cell infusion contains heparin in the infusate. Enoxaparin (LOVENOX®), a low molecular weight heparin, is initiated more than 4 hours but less than 12 hours after transplant, using 30 mg subcutaneously every 12 hours for 14 days.

Management of Adverse Events. Complications after the Islet Cell Infusion. Postoperative bleeding necessitates close hemodynamic monitoring, and as needed in the intensive care unit. Blood transfusion reversal of anticoagulation and the need for invasive procedure are decided upon by the surgical team taking care of the patient.

If portal vein thrombosis occurs, anticoagulation using Enoxaparin is prolonged for three months and follow-up imaging is arranged as indicated. Portal vein thrombosis—partial or complete—is a contraindication for repeat islet infusion.

Elevation of Liver Enzymes. LFT abnormalities are common (93% of patients) with a peak rise 3-4 days after transplantation.

Dose Adjustments of Sirolimus (SIR) and PROGRAF® (TAC) Due to Adverse Events. The Sirolimus (SIR) or PROGRAF® (TAC) dose should be increased or decreased to achieve the targeted whole blood concentrations in the absence of unacceptable toxicity or rejection. In the event of adverse events, toxicity should be first managed by lowering the SIR and TAC dose so that SIR/TAC levels are at the lower end of the desired target range. Lower target levels may be used if toxicity persists and must be treated.

Dose Adjustments of CELLCEPT® (MMF) Due to Leukopenia. The CELLCEPT® (MMF) dose may be decreased as needed for patients with leukopenia.

Dosage Adjustments of Sirolimus (SIR), PROGRAF® (TAC) and CELLCEPT® (MMF) Due to Gastrointestinal Toxicity. Symptoms of gastrointestinal toxicity including nausea, vomiting, diarrhea, and abdominal pain requires a decision whether to alter SIR, TAC and/or MMF dosing. The decision should be based on several factors including: nature and severity of toxicity and TAC level.

Diarrhea may be treated as follows:
1. Exclude infectious causes (Clostridium difficile and enteropathogens) and treat if necessary.
2. Administer SIR, TAC and MMF separately at different times (preferably 2 hours apart).
3. Determine TAC level and adjust dose to maintain near the lower level of the desired target range.
4. If SIR and TAC levels are near the lower end of the desired therapeutic range and infectious causes of diarrhea have been excluded, administer agents such as Lomotil or tincture of opium to decrease diarrhea so that the immunosuppressant dosing can be maintained.
5. If diarrhea persists, finally, reduce the CELLCEPT® dose by 250-500 mg/day increments and consult Principal Investigator for further treatment management. Consider the use of Myfortic as a substitute for CELLCEPT.

If a patient's immunosuppressive regimen is altered in order to manage an adverse event, the patient should be returned to their previous baseline immunosuppressive regimen as soon as the adverse event has resolved. All dose adjustments of immunosuppressive medications must be recorded on the patient's Case Report Form.

Drug Supplies, Accountability, Storage, Reconstitution/Dilution and Administration. The medications which are part of the research protocol may be supplied by the Pharmacy at Baylor University Medical Center in Dallas, Tex. or Baylor All Saints Medical Center in Fort Worth, Tex. Preparation may be performed by pharmacy standards. Drug administration and records as inpatients will follow the nursing staff orders in place. The nursing staff and the investigators will ensure the proper patient education regarding the medications was delivered before the patient is discharged from hospital.

Treatment Compliance. Compliance may be assured by having the immunosuppressive medications administered under the direction of the investigator and/or designated staff members while the patient remains hospitalized. Compliance may be monitored by trough level monitoring at each patient visit. Whole blood levels of TAC will verify that patients are maintaining the regimen prescribed.

Concomitant Medications and Therapies. Cytomegalovirus Prophylaxis. Administer oral valganciclovir for minimum of 14 weeks irrespective of the donor and the recipient's cytomegalovirus serology status in order to protect from future lymphoprolipherative disorders or graft loss. Any FDA-approved alternative therapy may be utilized in the event that valganciclovir is unavailable.

Pneumocystis carinii Pneumonia Prophylaxis. A standard Pneumocystis carinii pneumonia prophylactic regimen per institutional protocol should be given uniformly to all treatment groups for duration of the study.

Bacterial Prophylaxis. Pre-operative bacterial prophylaxis should be given using:
Vancomycin 500 mg IV pre-transplant and another 2 doses every 12 hours for 24 hours;
Merrem 500 mg IV pre-transplant and continued 3 doses every 8 hours for 24 hours. Miscellaneous

Enteric coated aspirin (81 mg per day) to be started on day 7 post transplant. Aspirin may be stopped for any subsequent infusion scheduled, and restarted on day 7 again post-transplant. Vitamin A (25,000 IU per day), Vitamin B₆ (100 mg per day), and Vitamin E (800 IU per day) may be administered orally for one month. Ulcer prophylaxis will also be administered (40 mg per day) to be started day 1 post-transplant.

Prohibited Medications, the following medications are not permitted in the protocol:
Basiliximab (SIMULECT®)
Corticosteroids
Cyclosporine
Terfenadine, astemizole, pimozide, ketoconazole—must be discontinued before rapamycin is initiated
St. John's wort
Fluconazole is prohibited for prophylaxis of oral candidiasis. It can be used for treatment of Candida infections up to 2 weeks with close monitoring of SIR and TAC levels
The use of cytochrome P-450 inducers or inhibitors should be avoided unless considered essential treatment by an investigator and approved by the principal investigator.

Schedule and description of auxillary study procedures. Screening procedures. Patients who have been identified for pancreatic islet cell transplantation may be screened for the inclusion/exclusion criteria. The patient's eligibility may be documented on an eligibility case report form. The following baseline evaluations must be completed prior to study enrollment:
Obtain signed and dated informed consent.
Physician will perform clinical exam
Record donor and recipient serological status for Hepatitis B and C, Human Immunodeficiency Virus (HIV), and Human T-Cell Lymphotropic virus 1 (HTLV-1).
Record donor and recipient serological status for Cytomegalovirus (CMV), and Epstein-Barr virus.
Perform urine or serum pregnancy test (on admission to hospital) on women who are of childbearing potential.
Obtain medical history prior to transplant. Include diagnosis for transplant, secondary diagnoses concomitant medications, and pre-study medications taken up to 7 days prior to transplant.
Obtain height and weight.
Record insulin requirements (product and dosage), blood glucose levels, adverse events, and hypoglycemic episodes.
Perform laboratory evaluations:
   CBC (hemoglobin, hematocrit, WBC with differential and platelet count)
   Coagulation tests: PT, INR, PTT
   Serum Chemistries: serum creatinine, BUN, Mg, phosphorus, Na, K, albumin, calcium, and glucose
   Serum amylase and lipase
   Thyroid hormone profile (T4, TSH and Free T4)
   Hepatic Profile: total bilirubin, AST, ALT, and alkaline phosphatase
   Lipid profile
   Hemoglobin A_{1C} and C-peptide.
   Urinalysis and urine culture
   24 hour urine for microalbumin
   Glofil
   DNA microarray, Auto-antibody, Epimax, ImmuKnow
MAGE determination

One Day Prior to Transplant (Day −1)

On the day before the transplant (Day −1), patients who have screened (See sections 7.1) may be tested again for the following to ensure continued eligibility for the study. Obtain signed and dated informed consent.
Record donor serological status for CMV and EBV
Confirm inclusion and exclusion criteria
Physician will perform clinical exam
Perform urine or serum pregnancy test (on admission to hospital) on women who are of childbearing potential.
Obtain medical history prior to transplant. Include diagnosis for transplant, secondary diagnoses concomitant medications, and pre-study medications taken up to 7 days prior to transplant.
Obtain height and weight.
Record insulin requirements (product and dosage), blood glucose levels, adverse events, and hypoglycemic episodes.
Administer medication as described in section 6
Perform laboratory evaluations:
   CBC (hemoglobin, hematocrit, WBC with differential and platelet count)
   Coagulation tests: PT, INR, PTT
   Serum Chemistries: serum creatinine, BUN, Mg, phosphorus, Na, K, albumin, calcium, and glucose
   Serum amylase and lipase
   Thyroid hormone profile (T4, TSH and Free T4)
   Hepatic Profile: total bilirubin, AST, ALT, and alkaline phosphatase
   Lipid profile
   Hemoglobin A_{1C} and C-peptide.
   Urinalysis and urine culture
   PRA.
   DNA microarray, Auto-antibody, Epimax, ImmuKnow

Study Procedures During Treatment

The following procedures may be performed and the data recorded as described on the following days. Day of Transplant: For patients who have consented, have met the inclusion/exclusion criteria, and have had a pancreatic islet cell transplant, the Day zero (Day of Transplant) procedures may be performed:
Measure and record portal venous pressures (intra-procedurally)
Administer medication as described in section 6
Obtain transplant information including date of transplant and cold ischemia time
Record recipient age, gender, and race
Record donor age, gender, and race
Record specified insulin requirements (product and dosage), blood glucose levels, hypoglycemic episodes, immunosuppressive drug doses, concomitant medications, adverse events, and weight.
Perform laboratory evaluations:
   CBC (hemoglobin, hematocrit, WBC with differential and platelet count) q8 hrs after transplant for 24 hours.
   PT, INR, PTT.
   Serum Chemistries: creatinine, BUN, Mg, Na, K, phosphorus, albumin, calcium, and glucose.
   Hepatic Profile: total bilirubin, AST, ALT, and alkaline phosphatase q8 hrs after transplant for 24 hours.
   C-peptide.
   Amylase q8 hrs after transplant for 24 hours.
   Lipase
   Anakinra: 100 mg subcutaneous injection immediately pre-transplant
   Etanercept: 50 mg intravenous injection immediately pre-transplant

Day 1 Post-Transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival.
Physician will perform patient clinical exam including weight.
Perform laboratory evaluations:
   CBC (hemoglobin, hematocrit, WBC with differential and platelet count) q8 hrs after transplant for 24 hours.
   PT, INR, PTT
   Serum Chemistries: creatinine, BUN, Mg, Na, K, albumin, phosphorus, calcium, and glucose
   Hepatic Profile: total bilirubin, AST, ALT, and alkaline phosphatase q8 hrs after transplant for 24 hours.
   Amylase q8 hrs after transplant for 24 hours.
   Lipase
   C-peptide
   DNA microarray, Auto-antibody
Tacrolimus: trough level, obtained 10-12 hours after oral dose
Rapamycin trough level
Doppler ultrasonogram of the liver.
Anakinra: 100 mg subcutaneous injection

Day 2 and 4 Post-Transplant:
Record immunosuppressive drug doses, concomitant medications, adverse events, hypoglycemic episodes, and opportunistic infections.
Thymoglobulin may be given intravenously at a dose of 1.5 mg/kg.
Record insulin requirements (product and dosage) and blood glucose levels.
Perform clinical assessment for graft survival
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   Hepatic Profile: total bilirubin, AST, ALT, and alkaline phosphatase
   C-peptide
   DNA microarray, ImmuKnow
Anakinra: 100 mg subcutaneous injection
Tacrolimus: trough level, obtained 10-12 hours after oral dose
Rapamycin trough level.

Days 3 and 5 Post-Transplant:
Record immunosuppressive drug doses, concomitant medications, adverse events, hypoglycemic episodes, and opportunistic infections. Information may be obtained from patients via phone or e-mail.
Record insulin requirements (product and dosage) and blood glucose levels. Information may be obtained from patients via GlucoMON. The patient is required to have a written log as a backup.
Etanercept: 25 mg subcutaneous injection (day 3)
Anakinra: 100 mg subcutaneous injection

Day 6 Post-Transplant:
Record immunosuppressive drug doses, concomitant medications, adverse events, hypoglycemic episodes, and opportunistic infections.
Thymoglobulin may be given intravenously at a dose of 1.5 mg/kg.
Record insulin requirements (product and dosage) and blood glucose levels.
Perform clinical assessment for graft survival
Physician will perform patient clinical exam including weight.
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   Hepatic Profile: total bilirubin, AST, ALT, and alkaline phosphatase
   C-peptide
   DNA microarray, Auto-antibody, ImmuKnow
Anakinra: 100 mg subcutaneous injection
Etanercept: 25 mg subcutaneous injection (day 7)
Tacrolimus: trough level, obtained 10-12 hours after oral dose
Rapamycin trough level

Day 10 Post-Transplant
Etanercept: 25 mg subcutaneous injection
Thymoglobulin

Day 14 Post-Transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Physician will perform patient clinical exam including weight.
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   C-peptide
   DNA microarray, Auto-antibody, ImmuKnow
Tacrolimus: trough level, obtained 10-12 hours after oral dose
Rapamycin trough level.

Day 21 Post-Transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Physician will perform patient clinical exam including weight.
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   Hepatic Profile: total bilirubin, AST, ALT, and alkaline phosphatase
   C-peptide
   HbA1c
   Urinalysis
   DNA microarray, Auto-antibody, ImmuKnow
Tacrolimus: trough level, obtained 10-12 hours after oral dose
Rapamycin trough level
Doppler ultrasonogram of the liver.
Ensure® Challenge
MAGE determination

Day 28 Post-Transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Physician will perform patient clinical exam including weight.
IVGTT
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   C-peptide
   Lipid profile
   DNA microarray, Auto-antibody, ImmuKnow
Tacrolimus: trough level, obtained 10-12 hours after oral dose
Rapamycin trough level.
PRA.

Every Two Weeks during the Second Month, and Monthly Thereafter Until the End of the Study:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Physician will perform patient clinical exam including weight.
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   HbA1c (monthly)
   C-peptide
   Lipid profile (every month for the first 6 months, every other month after 6 months and every three months after the first year from the initial transplant)
   PRA (monthly)
   DNA microarray, Auto-antibody, ImmuKnow
Tacrolimus: trough level, obtained 10-12 hours after oral dose
Rapamycin trough level.
MAGE determination second month.

Monthly Three Months Post-transplant Until Two Years Post-transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   HbA_{1C} (monthly)
   C-peptide
   PRA. (monthly)
   DNA microarray, Auto-antibody, ImmuKnow
Tacrolimus: Trough Level, obtained 10-12 hours after oral dose
Rapamycin trough level.
MAGE determination.

At 3, 6, 9, 12, 18, 24 Months Post-transplant:
Record immunosuppressive drug doses, concomitant medications, adverse events, hypoglycemic episodes, and opportunistic infections.
Record insulin requirements (product and dosage) and blood glucose levels.
Perform clinical assessment for graft survival
Physician will perform patient clinical exam including weight (every 3 months)
Perform laboratory evaluations:
   CBC (hemoglobin, hematocrit, WBC with differential and platelet count)
   Serum Chemistries: creatinine, BUN, Mg, Na, K, albumin, phosphorus, calcium, and glucose
   C-peptide
   Hepatic Profile: total bilirubin, AST, ALT, and alkaline phosphatase (every 3 months)
   HbA1c (monthly)
   Lipid profile (every month the first 6 months, every other month after 6 months and every three months after the first year from the initial transplant)
   Urinalysis (every 3 months)
   24 hour urine collection for microalbumin (Months 12 and 24)
   Glo-fil (Months 3, 6, and 18)
   IVGTT (6 months)
   PRA. (monthly)
   DNA microarray, Auto-antibody, ImmuKnow
   Epimax (Months 3 and 9)
Tacrolimus: Trough Level, obtained 10-12 hours after oral dose
Rapamycin trough level
Doppler ultrasonogram of the liver
MAGE determination
Patients will complete a self-administered “Quality of Life” questionnaire
Assessment of primary and secondary efficacy parameters
Ensure Challenge (Months 3, 12, 18 and 24)
Eye exam (Months 6, 12, 18, and 24) to assess for eye abnormalities caused by diabetes, such as diabetic retinopathy. Exams may be performed by external ophthalmologist.

Other Scheduled Visits. In the event of a second islet cell infusion, the scheduled procedures may be similar to those performed with the first transplant. Four weeks after the second transplant the patient will re-enter into the initial schedule according to the initial timeframe.

Unscheduled Additional Visits. If the patient requires treatment between scheduled visits, all data related to the treatment may be added to the patient file.

Definitions and detailed descriptions of assessments and endpoints.

Safety. The assessment of safety may be based upon adverse events, opportunistic infections, malignancies, and medically significant changes in laboratory values or imaging studies. In the event that a patient experiences an adverse event, the investigator may be asked to rate causality to the study procedure and/or medication and the event(s) related to the treatment should be recorded on the adverse event case report form.

Adverse Events. Definition. An adverse event (AE) is any reaction, side effect, or other untoward medical occurrence that is temporally, but not necessarily causally, related to the procedure (islet cell transplant) or to the medications or treatments related to the procedure. For the purposes of this study, the following additional adverse events may be defined for uniform reporting.

Bleeding. Any episode that correlates a drop in Hb of more than 2 g/dL after procedure with evidence of bleeding by abdominal imaging (ultrasonography or computed tomography scan) may be recorded and treated as a Grade 3 adverse event. Bleeding necessitating blood transfusion represents a SAE.

Portal Vein Thrombosis. Formation of clot in the portal vein or one of its branches (occlusive or non-occlusive) as demonstrated by Doppler ultrasonography may be recorded as thrombosis of that venous structure. A partially occluding thrombus that shows some flow limitation but where normal directional flow is preserved in a branch of the portal vein may be recorded as a Grade 2 adverse event. A partial or total thrombus of a branch portal vein that results in reversal of flow may be recorded as a Grade 3 adverse event. A thrombus of the main portal vein, whether it resulted in flow reversal or not, may be recorded as a Grade 3 adverse event.

Bacterial, Viral, and Fungal Infections. Bacterial, viral and fungal infections and other opportunistic infections may be recorded. Infections may be defined as any of the following that requires hospitalization and treatment with an antimicrobial, anti viral or antifungal agent (not prophylaxis):
Positive cultures from a normally sterile site.
Pathologic identification of microbial agents.
Significant serologic changes related to clinical symptoms.
Typical clinical presentation of disease/infection documented by investigator or appropriate consultant.

An increase in the number and/or severity of infections over what is reasonably expected in these patients may be treated as a Grade 3 adverse event.

Hepatotoxicity. Diagnosis of hepatotoxicity is only considered if biochemical changes are confirmed histologically and all other diagnoses are excluded (i.e., portal vein thrombosis, hematomas of the liver, viral hepatitis, etc.). Hepatotoxicity must be diagnosed by biopsy and differentiated from rejection, viral hepatitis, etc. If hepatotoxicity is confirmed by biopsy, the primary investigator must determine if it is related to the immunosuppressive agents. The principal investigator should be contacted for consultation regarding discontinuation of the drug(s). In all cases of hepatotoxicity, TAC trough and sirolimus levels should be drawn and sent for analysis.

Other Adverse Events: Other adverse events may be recorded including:
Leukopenia defined as a WBC <3,000 μL
Anemia defined as hemoglobin <9 mg/dL
Thrombocytopenia defined as a platelet count <50,000 μL
Neutropenia (ANC <500/uL)
Malignancies, lymphoma and lymphoprolipherative disease. Development of any post transplant malignancy occurring during the study may be evaluated up to and at Day 730.
Other adverse events that are protracted and not seen as part of normal post-transplant recovery.

Reporting of Adverse Events. All adverse events, whether ascertained through patient interviews, physical examination, laboratory findings, or other means are to be recorded. The association with the study procedure or medications may be noted. Each adverse event is to be recorded on an adverse event case report form. The investigator will provide date of onset and resolution, severity, relatedness to the study procedure/drugs, action(s) taken, changes in immunosuppressant dosing or accompanying medications, and outcome. Adverse events ongoing at the final visit may be followed up for as long as necessary to adequately evaluate the patient's safety or until the event stabilizes. If the event resolves during the study or follow-up period, a resolution date should be documented on the case report form. Once adverse events have resolved, attempts should be made to return the patient to their baseline therapies.

Stopping Rules. Adverse events and may be graded as mild (grade 1), moderate (grade 2), severe (grade 3) or life threatening (grade 4). Because of the patients' underlying disease, hypoglycemic and hyperglycemic toxicities will not be included in the stopping rules; however, they will still be included in study reports. Elevations in one or more liver enzymes may be graded as a single adverse event. The following adverse events (as described above) may be treated as Grade 3 adverse events: Bleeding, Portal vein thrombosis and an increase in the number and/or severity of opportunistic infections.

Any single grade 3 adverse event may be discussed with the production and clinical islet transplant teams. If the event is self-limiting with little or no clinical significance, the trial may be continued. However, any single grade 4 adverse event or two or more grade 3 adverse events will result in immediate cessation of further transplants and notification of both the FDA and IRB. The trial will remain halted until approval to resume is obtained from the FDA. A full reporting of all adverse events may be made to both FDA and IRB on a yearly basis. Serious Adverse Events. Definition. A serious adverse event (SAE) is any adverse event (AE) occurring that results in any of the following outcomes:
1. Death.
2. Life-threatening AE.
3. Inpatient hospitalization or prolongation of existing hospitalization related to the procedures or the immunosuppression treatment.
4. Persistent or significant disability or incapacity.
5. Congenital abnormality or birth defect.
6. Important medical event that would potentially result in any of items 1-5.

Life threatening means that the study participant (subject/patient) was, in the opinion of the investigator, at immediate risk of death from the reaction as it occurred. An important medical event is any medical event that may not result in one of the above outcomes, but may jeopardize the health of the study participant (subject/patient) or require medical or surgical intervention to prevent one of the outcomes listed in the above definition of SAEs. Any other event thought by the investigator to be serious should also be reported.

The term “severe” is used to grade intensity and is not synonymous with the term “serious”.

Hospitalization or prolongation of hospitalization for elective surgical procedures unrelated to the transplant or the associated treatment need not be recorded.

Reporting. In the event of an SAE, including death, the investigator (or coordinator) must contact the principal investigator and the IND sponsor immediately (i.e. within 24 hours of the awareness of the event by the investigator or coordinator) by telephone. A Serious Adverse Event Worksheet should be used to transmit written SAE information.

Follow-up information for the event provided to the principal investigator within 7 days as necessary. All pregnancies must be reported to the IRB/FDA in the same manner as a SAE. The IND sponsor will submit IND Safety Reports to the FDA within 15 calendar days (7 days for fatal or life-threatening events) after becoming aware of the event and other regulatory agencies as necessary, and will inform the investigators of such regulatory reports. Investigators must submit safety reports as required by the Institutional Review Board (IRB). Documentation of the submission to and receipt by the IRB should be retained. A MedWatch, or equivalent, report may be filed as needed.

Investigators may contact the Principal Investigator for any other problems related to the safety, welfare, or rights of the study participant (subject/patient).

Pregnancy. Although not an adverse event, given the potential implication of the study on the outcome of the fetus, pregnancy is reported the same as a serious adverse event. All the reports on pregnancy must be followed for information regarding the course of the pregnancy and delivery, as well as the condition of the infant. Follow-up information regarding the course of the pregnancy and delivery, as well as the condition of the infant should be provided to the principal investigator in a timely manner.

Definition of Endpoints. Sustained euglycemia with or without exogenous insulin. Achievement of glucose control (serum glucose range: 80-120 mg/dL) with HbA1c below 7% and C-peptide levels above 0.5 ng/dL without the use of insulin for 2 months.

Hypoglycemic Unawareness. Clinical picture of cognitive loss in the context of serum glucose below 50 mg/dL.

Reduced Insulin Requirements. Any reduction in the daily insulin requirements of 50% as compared to the pre-transplant insulin needs.

Graft Function and Graft Loss. Graft function is assumed if there is a detectable stimulated serum C-peptide level above the pre-transplant level. A return of the stimulated serum C-peptide level to baseline or zero signifies graft loss.

Efficacy. An assessment of the efficacy of the islet cell transplantation procedure may be based on the following parameters:

Primary Endpoint. To assess the safety of islet transplantation and to assess the 12-month and 24-month post-final transplant endpoint in patients who underwent pancreatic islet cell transplantation. The endpoint will consist of the restoration of sustained euglycemia without exogenous insulin or with reduced insulin dosage.

Patients who do not meet these criteria will continue to be followed for the duration of the study for safety assessment and secondary endpoint.

Secondary Endpoint. Absence of hypoglycemic unawareness as defined by: Clinical picture of cognitive loss in the context of serum glucose below 50 mg/dL.

Additional assessments, Data may be collected and analyzed to assess the following:
Incidence of hypoglycemia episodes.
Insulin requirements in patients who did not become insulin independent.
The total islet mass needed to achieve sustained euglycemia with or without exogenous insulin.
The number of islet cell infusions needed to achieve sustained euglycemia with or without exogenous insulin.
The islet cell mass, its viability and function obtained after transport using the two-layer preservation method, remote site processing and islet culture.
Renal function.
Morbidity related to the immunosuppression regimen.
Morbidity related to the islet cells infusion.
Quality of life of the recipients.

The above data may be analyzed in conjunction with transport method, transport media, cold ischemia time islet dose and cell culture time/temperature to determine the ideal criteria for islet cell transplant.

HYPO score. Hypoglycemia may be assessed by HYPO score described by Ryan et al. before and after islet transplantation yearly³³. This score is a measure of the degree of hypoglycemic unawareness experienced. Low scores reflect little to no hypoglycemic unawareness. The use of this score will help assess the efficacy of islet cell transplantation in reducing/eliminating hypoglycemic unawareness.

m-value. An m-value may be used to assess the stability of blood glucose levels.

The average of 6 blood glucose measurement (before and after breakfast, lunch and dinner) may be used. The formula of individual m-value is =1(10×log₁₀ [blood glucose (mg/dl)/100])³1, Average of 6 measurements may be taken for a daily m-value.

Immune Testing. The research regarding immunological profile before and after islet transplantation has been limited. In this study, we will perform whole genome expression using DNA microarray and cytokine profile analysis using Epimax techniques, both of them were developed at the Baylor Institute of Immunology Research. For auto-antibody assay, GAD 65, IA-2, insulin and Znt8 may be measured at the University of Colorado Health Science Center (PI, Dr. George Eisenbarth). For immune function assay we will use Cylex Immune Cell Function Assay.

For microarray, 3 ml of blood may be collected before and after islet transplantation as indicated hereinbelow. Blood may be examined with microarray for analyzing changing pattern of gene due to type 1 diabetes, islet transplantation and immunosuppressive drug regimen. This will identify immunological events linked with transplant rejection and provide metrics for adjusting immunosuppressive regimen.

For Epimax, 30 ml of blood may be collected before (at the time of initial screening and listing) and after islet transplantation (post-operative day 30 and 90) therefore total 4 time points. Blood may be examined with Epimax technique for analyzing the changing pattern of cytokine due to type 1 diabetes and islet transplantation.

For auto-antibody assay, 1 cc of blood may be collected before and after islet transplantation as indicated in appendix A. Blood may be spun and collect 300 microL serum and kept in freezer until shipping. The serum may be sent to the University of Colorado Health Science Center. Recently we found multiple positive auto-antibodies correlated with poor outcome due to possible recurrence of auto-immune disease. Therefore it is important to know whether the patients have auto-antibody.

The Cylex Immune Cell Function Assay (ImmuKnow assay) was cleared by the FDA for detection of cell-mediated immunity in an immunosuppressed population. Recently we used Cylex Immune Cell Function Assay after liver transplantation for identifying rejection or infection with excellent clinical outcome. Cylex Immune Cell Function Assay should be especially useful after islet transplantation, since biopsy is almost impossible for detection of rejection or infection. 3 cc of blood samples may be collected for Cylex immune cell function before and after islet transplantation as indicated in appendix A.

Study Withdrawal Criteria. Patients may be withdrawn from the study due to the following reasons:
Patient withdraws consent.
Investigator believes it is no longer in the best interest of the patient to remain in the study due to safety or efficacy issues.
Patient becomes pregnant.
Patient is lost to follow up.
Patient necessitates immunosuppression treatment prohibited by the study regimen.

Patients who are withdrawn from the study will continue to be followed for the entire 24 months duration of the study. Reasonable attempts may be made to find subjects lost to follow-up.

Termination of Study. The principal investigator shall have the right to terminate this study at his discretion with written notice to the IRB and FDA.

Possible reasons for termination of the study include, but are not limited to:
1. Unsatisfactory enrollment with respect to quantity or quality.
2. Incidence and/or severity of adverse events in the study that indicate a potential health hazard caused by treatment with the investigational procedure.

In all events, a final examination must be performed on each subject who is still in the study at the time of termination as well as on any patient who is terminated prematurely for any reason. The investigator will enter the data on the case report form as complete as possible. At the end of the study, the principal investigator will then collect all study materials.

All patients will continue to be followed for safety assessment after the termination of the study for at least 24 months.

Care after completion of the Study. Two years after the final islet transplantation, the patients will receive continuous follow up at Baylor. Transplanted islets may deteriorate in function. In such cases, we plan supplemental islet transplantation. Risks and benefits of the study. The potential risks for patients involved in the study are related to the islet cell infusion and/or the accompanying treatment. Risks of Islet Cell Infusion. Risks of the Infusion Procedure. The islet cell infusion procedure is performed through a catheter inserted in the portal vein percutaneously using radiologic guidance, under intravenous sedation.

The most common complication of the procedure is bleeding from the puncture site, either intra-abdominal or intrahepatic, either subcapsular or intraparenchymal. While minor bleeding episodes are frequent, they are clinically apparent in 14% of cases and 2% necessitate blood transfusion. Surgery for hemostasis was reported in less than five cases worldwide. Other complications include gallbladder puncture (2%), hemobilia (1%), biliary leaks (1%). Thrombosis of a portal vein branch is possible in 2% of cases. Follow-up of this shows recanalization of the affected veins and there were no reports of permanent damage from thrombosis. The risk of thrombosis of the main portal vein is less than 0.5%. It has been reported in combined liver and islet transplantation but not in an islet cell transplantation procedure.

Hemothorax or pneumothorax is exceedingly rare. Liver abscess formation is theoretically possible, but has not been reported so far. Pain is common during the procedure, due directly to the procedure or due to the rise in the portal vein pressure. Pain is uncommon after the procedure is over. Conscious sedation used with the procedure could involve the risks of respiratory depression or arrest, and patients monitored closely while in the Radiology suite and treated as necessary.

The transplant procedure involves radiation exposure from catheter placement and portography. There may also be radiation exposure from several standard tests such as chest x-rays as clinically indicated. The total radiation dose for the two-year protocol is expected to be less than the maximum one-year radiation exposure for professionals working with radiation. This amount is 10 to 15 times the background radiation exposure per one year.

Other Risks of the Infusion. Temporary rise in the liver function tests are common (up to 93% of cases), and up to 46% of them are significant. This usually will return to normal within two weeks of the transplant and do not have impact on the liver function long-term.

Another possible risk of islet transplantation is the introduction of infection with the islets. This should be substantially reduced by the appropriate screening of donors for infection, prophylactic antibiotics administered to the recipient, and by microscopic examination of the islet preparation for presence of bacteria. Although islet preparations may be assessed by culture, the results will usually not be available until after the transplant has been completed. However, this information may be important to dictate treatment if indicated.

Islet cell preparations can transmit viral infections, such as hepatitis B or C, HIV, HTLV, and CMV. The risk of transmission is extremely low given the donor selection process and the fact that the abovementioned viruses are not known to be carried by islet cells.

Transplantation of allogeneic tissues including pancreatic islets, whether successful or unsuccessful, may induce an immune response in the recipient and generate cytotoxic antibodies against donor HLA antigens. This occurs with solid organ transplantation as well, and it can become a problem in the event the subject needs a future transplant.

Risks of Immunosuppression Medication. Chronic immunosuppression carries a general risk of opportunistic infection and a small but discernible risk for development of malignancy. In solid organ recipients registry data has identified that three cancer types are of increased incidence over that of the general population: vulvar carcinoma, non-melanoma skin cancer, and lymphoma. No islet cell recipient has been reported to have developed a malignancy.

The risk of lymphoma is estimated at 0.5% to 1% in adult solid organ transplant recipients, although no cases of lymphoma have been reported in more than 500 islet transplant patients under the Edmonton protocol or any of its derivatives.

Transplant recipients of islet cells, like those of solid organs, are generally at a higher risk of developing infections than the general populations, and at higher risk of these infections becoming more severe than in the general population. Examples of such common infections include bacterial, viral, or fungal pneumonia, bacterial or fungal urinary tract infections, cytomegalovirus infections, Pneumocystis carini infections, and commonplace viruses such as the common cold. To date, no islet cell recipients have died of infection. Some at-risk infections, notably CMV and Pneumocysitis, are specifically prophylaxed against and have not been reported in these patients. Given that type 1 diabetics are also at higher risk of infections than the general population, the increased risk of infections for the islet patients is likely smaller that the increased risk of a non-diabetic patient (general population) would incur.

Each immunosuppressant used also carries specific side effects as detailed below:

Etanercept: The possible side effects for Enbrel® include rare occurrences of susceptibility to serious infections, nervous system diseases, lack of production of sufficient quantity of blood cells, heart problems and allergy reactions.

Other more common side effects include: skin redness, rash, swelling, itching or bruising at the site of injection, upper respiratory tract infections and headaches.

Tacrolimus: The most frequent side effects of Prograf® are: tremor, headache, diarrhea or constipation, abdominal pain, hypertension, renal function impairment, and insomnia.

Other side effects that can occur include: paresthesia, hypo- or hyperesthesia of the extremities, nausea and vomiting, hypomagnesemia, anemia, muscle weakness, shortness of breath, extremity edema, skin rash or itching, non-specific pain, drug fever.

Sirolimus: The most common side effect of sirolimus is the occurrence of mouth ulcers (up to 85%). While typically self-limiting, some patients need to discontinue treatment because of them. Other common side effects include: hypercholesterolemia and hyperlipidemia (necessitating lipid lowering medications), thrombocytopenia and leukopenia, anemia, pneumonitis, hypokalemia, edema, skin rash, liver enzyme elevations, headache, diarrhea, other digestive symptoms, bone and joint pain. Although extremely rare, Rapamycin may cause an allergic reaction.

Anakinra (KINERET®): The most common side effect of anakinra is a reaction at the injection site, including redness, swelling, inflammation, and pain. These reactions usually disappear after the first month. Other side effects may include: Abdominal pain, bone and joint infections, diarrhea, flu-like symptoms, headache, nausea, serious infections such as cellulitis and pneumonia, sinus inflammation, upper respiratory infections.

Anakinra has been associated with an increased incidence of serious infections vs. placebo when used in combination with etanercept. These studies were conducted for up to 28 weeks, whereas patients in this study will receive these two drugs for a total of 10 days and may be closely monitored for infections.

Anti-thymocyte Globulin (Rabbit) (THYMOGLOBULIN®): The most common side effects associated with ATG include fever and chills. To a lesser extent, people have also experienced diarrhea, headache, aches/pains, nausea, swelling of extremities, shortness of breath, weakness, increased pulse and increased blood pressure.

Risks of Other Medication Used in the Study.

Heparin: Can increase the risk of bleeding from the liver puncture site, easy bruising, hematomas, thrombocytopenia. Additional risks of heparin or low-molecular weight heparin (Lovenox) include elevated liver function tests and thrombocytopenia directly caused by the formation of an anti-platelet antibody. The incidence of heparin-induced thrombocytopenia is estimated to be lower if low-molecular weight heparin (Lovenox) is used and is felt to be 0.2% in that setting.

Sitaglipin (JANUVIA®): The most common side effects reported with sitaglipin are: upper respiratory tract infection, stuffy or runny nose, sore throat and headache. sitaglipin may occasionally cause stomach discomfort and diarrhea. In studies, these side effects usually were mild and did not cause patients to stop taking sitaglipin. Other side effects not listed above may also occur.

Other Risks. Psychological impact: Clinical islet transplantation, as a potential therapy for Type I Diabetes Mellitus, has been discussed in the media and diabetes lay publications with a degree of optimism that is not justified on the basis of clinical results to date. Therefore, failure of the procedure to reverse hyperglycemia and maintain sustained euglycemia with or without exogenous insulin could be associated with a level of psychological disappointment that might progress to clinical depression.

Study Benefits. A successful islet cell transplant provides a degree of euglycemic control impossible to achieve with exogenous insulin therapy. This includes the elimination of dangerous hypoglycemic events. Euglycemic control lowers the risk of microvascular complications of diabetes (such as nephropathy, retinopathy, neuropathy, cardiopathy) and even reverses some of the long-term complications. The greatest benefit occurs when the subjects become free from insulin injections. Subjects having sustained euglycemia, although still requiring exogenous daily insulin, will still benefit from the long-term effects of glucose control.

Subjects who do not achieve sustained euglycemia with or without insulin may still benefit from closer follow-up as part of the study than with their routine follow-up.

Risk/Benefit Ratio. The potential benefits of transplant induced long-term euglycemia with this protocol must be weighed against the otherwise unnecessary risk of immunosuppression with every patient on an individual basis. The potential morbidity or mortality of dangerous hypoglycemic events and other acute sequelae of major excursions in blood sugar in these patients will have to be greater risks to them than the islet transplant procedure, and the need for life-long immunosuppression.

Statistical methodology. Populations for Analysis. The patients enrolled in the study may be included in the analysis if they received at least one islet cell infusion. The analysis will include the patients who eventually dropped out of the study for certain parameters and timeframe analysis. We expect a 2/15 (13.3%) rate of dropout or non-evaluable patients, from our previous experience with studies of transplant patients.

Patients that were originally enrolled and transplanted under BB-IND 11731 and BB-IND 12916 may be analyzed separately from patients who receive transplants solely under this new protocol under BB-IND 12916.

Sample Size and Statistical Power. A sample size of 15 patients was selected as an adequate size to provide a preliminary estimate of sustained euglycemia with or without exogenous insulin. The sample size calculation was based on the comparison of the primary efficacy endpoint variables hemoglobin A1c and C-peptide under the following assumptions:
Power was assessed assuming a paired t test may be used to compare baseline to the follow-up
For hemoglobin A_{1C}, a change from 8.5 to 6.0 with a standard deviation of the difference of 0.83 was hypothesized.
For C-peptide, a change from 0.2 to 2.0 with a standard deviation of the difference of 1.13 was hypothesized.
A two-sided test may be performed at the 0.05 level of significance.
Statistical power for the comparison of parametric variables: 80%.

Based on these assumptions, as well as a possible dropout rate of 13%, a sample size of 13 patients may be adequate to have a minimum of 80% power to detect a difference in the hypothesized primary efficacy endpoints at follow-up when compared to the baseline parameters.

Primary Efficacy Parameters. The primary efficacy endpoint variable is the proportion of patients who have restoration of sustained euglycemia without exogenous insulin or with reduced insulin dosage at 3, 6, 12 and 24 months after final islet cell transplantation. Hypothesized primary efficacy endpoint failure rate: 20%.

The following data may be collected to assess insulin dependence: Hemoglobin Aic, MAGE, m-value and C-peptide as measures of diabetes control; Insulin requirements in patients who did not become insulin independent; the total islet mass needed to achieve sustained euglycemia with or without exogenous insulin.

SUITO index=1500×c-peptide/[blood glucose level (mg/dl)-63 (mg/dl)] at fast. Secondary Efficacy Parameters. Each of the following secondary efficacy parameters may be evaluated at 3, 6, 12, and 24 months post final transplantation. Hypothesized secondary endpoint failure rate: 15%.

Other Data to be collected include: Presence/absence of hypoglycemic unawareness; Incidence of hypoglycemia episodes (although numeric-interval of values may be used); The number of islet cell infusions needed to achieve sustained euglycemia with or without exogenous insulin (although numeric-interval of values may be used); Assessment of renal function; Morbidity related to the immunosuppression regimen; Morbidity related to the islet cell infusion; Assessment of the quality of life of the recipients to be collected by patient self-evaluation, via a “Quality of Life” questionnaire used by our group with other post-transplant patients34. Collecting patients' opinions on how to make islet transplantation more satisfactory to the patients.

Statistical Methods. Categorical variables may be analyzed using McNemar's test. Continuous data may be analyzed using repeated measures analysis of variance and Friedman's test when the normality assumption is violated. Follow-up pairwise comparisons may be performed using the Bonferroni multiple comparisons procedure at the 0.05 level of significance. Kaplan Meier estimates for patient and graft survival may be used.

Administrative and regulatory considerations. Prior to Initiation of the Study. The following may be provided to Baylor University Medical Center and/or Baylor All Saints Medical Center prior to enrollment of the first patient:
1. A completed Food and Drug Administration (FDA) 1572 form.
2. Curricula vitae and medical licenses for the Investigator and Sub-Investigators.
3. The “Investigator Signature” page of the protocol and any applicable amendment(s) signed and dated by the investigator.
4. An IRB membership list or IRB assurance number.
5. Written verification of IRB approval of protocol, amendments, if applicable, and informed consent in compliance with federal regulation 21CFR Part 50 and 21 CFR Part 56.
6. Written documentation of financial disclosure in compliance with federal regulation 21CFR Part 54.
7. Documentation of laboratory certification and normal reference ranges.

Institutional Review Board Approval. Prior to its implementation, this protocol, including any subsequent amendments, must be submitted to FDA and approved by an IRB constituted according to FDA regulations. Any further amendments of or deviations from the approved protocol must be submitted to FDA and approved by an IRB constituted according to FDA regulations.

Signed Informed Consent. The investigator, or designee, is obligated to obtain from each patient, or the patient's legally authorized representative, i.e., parent/legal guardian, a signed and dated IRB approved written Informed Consent prior to performing any protocol designated procedure.

Amendments and/or Changes to Informed Consent. Submission to FDA and written verification of IRB approval may be obtained before any amendment is implemented which affects patient safety or the evaluation of safety and/or efficacy. Modifications of the protocol that are administrative in nature do not require IRB approval but may be submitted to the IRB and FDA for information. If there are changes to the informed consent, written verification of IRB approval must be obtained.

Duties of the Investigator. The investigator is obligated to conduct this study in accordance with federal regulation 21 CFR 312.60-69 as specified on the signed form FDA 1572, applicable state laws, and the International Conference on Harmonization: Good Clinical Practice: Consolidation Guideline. The investigator is responsible for informing the IRB of any safety issues related to the study and the study procedures including reports of serious adverse events, if required, and all IND safety reports.

Monitoring the Study. The IND sponsor will hire a monitor to review the study, as frequently as is necessary to assure compliance with Good Clinical Practices and protocol procedures. Source documents may be verified to ensure accurate completion of case report forms and will review regulatory documentation located at the research site. Case Report Forms (CRFs) may be 100% source document verified on safety and efficacy variables only. On remaining variables, the monitor will source document verify 20% of the data. FDA representatives reserve the right to visit sites at any time.

A Data Safety Monitoring Board (DSMB) consisting of BUMC and external members will meet no less than two times per year to review the safety and efficacy data from this clinical trial.

Records of the Study. It is the investigator's responsibility to retain all records and documents pertaining to the conduct of the study for 2 years, after the procedure is licensed or the IND is withdrawn and the FDA has been notified. The investigator agrees to obtain principal investigator's agreement prior to disposal, moving, or transferring of any study-related records.

Data generated by the methods described in the protocol may be recorded in the patient's medical records and/or study progress notes. All data may be transcribed legibly on case report forms supplied for each patient. The investigator will agree to provide access to the office, clinic, laboratory, and/or hospital records of all patients entered in this study. Access and inspection of these records may be required by Baylor University Medical Center or Baylor All Saints Medical Center and the principal investigator. In addition, all records may be subject to inspection by officials of the Food and Drug Administration or other health authorities.

The investigators shall make accurate and adequate written progress reports to the FDA and IRB at appropriate intervals, not exceeding 1 year. The principal investigator shall make an accurate and adequate final report to the FDA and IRB within 3 months after completion or termination of the study.

Patient Privacy. Baylor University Medical Center and Baylor All Saints Medical Center affirm the patient's right to protection against invasion of privacy. Only a patient identification number will identify patient data retrieved by Baylor University Medical Center or Baylor All Saints Medical Center. However, in compliance with federal regulations, the investigator is required to permit Baylor University Medical Center or Baylor All Saints Medical Center and, when necessary, representatives of the FDA or other government agencies, as required, to review and/or copy any medical records relevant to the study.

Should access to medical records require a waiver or authorization separate from the patient's statement of informed consent, it is the responsibility of the investigator to obtain such permission in writing from the appropriate individual.

Publication of Results. The data obtained from this study may eventually be presented at professional scientific meetings and/or published in scientific journals. The identity of study subjects may be protected in all publications by using codes or numbers.

TABLE 2
Immunosuppression medication and adjustments.
SIROLIMUS DOSAGE ADJUSTMENT

| SIR Target | SIR actual | | |
|---|---|---|---|
| range | trough level | | Suggested action |
| 12-15 ng/mL | <6 | ng/mL | Administer the loading dose and the new |
| (first | | | maintenance dose together on the first day, |
| 3 month | | | then the new maintenance dose thereafter |
| after ICT) | 6-12 | ng/mL | Adjust dose according to formula #1 |
| | 12-15 | ng/mL | No adjustment necessary |
| | >15 | ng/mL | Decrease dose according to formula #1 |
| 7-10 ng/mL | <4 | ng/mL | Administer the loading dose and the new |
| (>3 months | | | maintenance dose together on the first day, |
| after ICT) | | | then the new maintenance dose thereafter |
| | 4-7 | ng/mL | Adjust dose according to formula #2 |
| | 7-10 | ng/mL | No adjustment necessary |
| | >10 | ng/mL | Decrease dose according to formula #2 |

Formula #1: Dosage adjustment for SIR target level of 12-15 ng/dL: NEW DOSE (mg) ^{a} = Current dose ^{b} (mg) × [16/SIR trough level ^{c} (ng/mL)]
Formula #2: Dosage adjustment for SIR target level of 7-10 ng/dL: NEW DOSE (mg) ^{a} = Current dose ^{b} (mg) × [16/SIR trough level ^{c} (ng/mL)]
Formula #3: Calculation formula for loading dose: SIR LOADING DOSE (mg) = 3 × [New maintenance dose(mg) − Current maintenance dose(mg)]
Notes:
^{a} Sirolimus exhibits dose proportionality from 1 to 12 mg/m²
^{b} As currently given
^{c} If the sirolimus trough level is less than the limit of quantification (1.5 ng/mL), then assume that the current trough level is 1.5 ng/mL for the sake of the calculation.

Sirolimus has a half-life of approximately 3 days. Therefore it takes 2 weeks to reach a new steady-state level after the change of dose. A loading dose is necessary if the trough levels are below half the lower target trough level desired.
Subjects should be treated for hypercholesterolemia and hypertriglyceridemia (defined in both as above 200 mg/dL) first with diet adjustment and standard medications. SIR dose adjustments should be considered if the treatment has been optimized.

TABLE 3
SIROLIMUS - TOXICITY GUIDELINES
Dose adjustments for drug toxicity are outlined below.

| Laboratory test | | |
|---|---|---|
| | SIR level >10 ng/mL | |
| SIR level ≦10 ng/mL | and <20 ng/mL | SIR level ≦20 ng/mL |

Platelet Count

| | | | |
|---|---|---|---|
| <75,000/mm³ | Repeat level within 48-72 hours, | Reduce current SIR | Reduce current SIR dose by |
| | consider holding SIR pending | dose by 25% | 50%, repeat level within 5-7 |
| | results and monitor thereafter | | days |
| <50,000/mm³ | Hold or discontinue SIR | Reduce current SIR | Reduce current SIR dose by |
| | | dose by 25-33% | 75%, repeat level within 5-7 |
| | | | days |

WBC

| | | | |
|---|---|---|---|
| <3000/mm³ | Repeat level within 5-7 days and | Reduce current SIR | Reduce current SIR dose by |
| | monitor | dose by 25% | 50%, repeat level within 5-7 |
| | | | days |
| <2000/mm³ | Hold or discontinue SIR | Reduce current SIR | Reduce current SIR dose by |
| | | dose by 25-33% | 75%, repeat level within 5-7 |
| | | | days |

Triglycerides*

| | | | |
|---|---|---|---|
| >750 mg/dL | Repeat level within 5-7 days and | Reduce current SIR | Reduce current SIR dose by |
| | monitor | dose by 25% | 50%, repeat level within 5-7 |
| | | | days |
| >1000 mg/dL | Discontinue SIR | Reduce current SIR | Reduce current SIR dose by |
| | | dose by 25-33% | 75%, repeat level within 5-7 |
| | | | days |

Cholesterol*

| | | | |
|---|---|---|---|
| >500 mg/dL | Repeat level within 5-7 days and | Reduce current SIR | Reduce current SIR dose by |
| | monitor | dose by 25% | 50%, repeat level within 5-7 |
| | | | days |
| >750 mg/dL | Discontinue SIR | Reduce current SIR | Reduce current SIR dose by |
| | | dose by 25-33% | 75%, repeat level within 5-7 |
| | | | days |

Dose reduction should be made after other causes of toxicity have been ruled out or treated. Questions regarding dose modifications should be addressed to the principal investigator.

Sirolimus and Tacrolimus: Clinically significant inducers and inhibitors of the cytochrome P-450.

SIR and TAC are a substrate of both cytochrome P-450 (CYP) and P-glycoprotein. It is extensively metabolized by the CYP3A4 isoenzyme in the gut wall and in the liver. Absorption and subsequent metabolism is influenced by drugs that affect this isoenzyme.

Drug Interactions:

Diltiazem—can increase SIR/TAC levels. If absolutely necessary, administration of diltiazem necessitates careful monitoring and SIR/TAC dose adjustments may be necessary.

Ketoconazole—can significantly raise SIR/TAC levels, therefore the use of ketoconazole should be avoided.

TABLE 4
OTHER DRUGS OR PRODUCTS THAT CAN INCREASE
SIR/TAC BLOOD CONCENTRATIONS:

| | | | |
|---|---|---|---|
| Calcium channel | Nicardipine | Verapamil | Nifedipine, |
| blockers | | | Diltiazem |
| Antifungal agents | Clotrimazole | Fluconazole | Itroconazole |
| Macrolide | Erythromicin | Clarithromycin | Troleandomycin |
| antibiotics | | | |
| Gastrointestinal | Metoclopramide | Erythromicin | Cisapride |
| prokinetic agents | | | |
| HIV protease | Ritonavir | Indinavir | Danazol |
| inhibitors | | | |
| Other drugs/ | Bromocriptine | Cimetidine | Metoclopramide |
| products | | | |
| Protease | Grapefruit juice | | |
| inhibitors | | | |

Rifampin—can substantially decrease SIR/TAC levels. Alternative therapeutic agents. should be considered.

TABLE 5
OTHER DRUGS THAT CAN DECREASE SIR/TAC LEVELS

| | | | |
|---|---|---|---|
| Anticonvulsivants | Carbamazepine | Phenobarbital | Phenytoin |
| Antibiotics | Rifabutin | Rifampin | Rifapentine |
| Other drugs/products | St. John's wort | | |

NOTE:
This list is not all-inclusive. Subjects should check with the investigators before starting or stopping any medication, including over the counter products. The patient's diet should be reviewed by the investigators in conjunction with the medications.

Sirolimus—Toxicity guidelines. If SIR is withheld because of laboratory abnormalities, it may be restarted if the laboratory values in question return to baseline and the dose has been held for no longer than 10 days. Subjects who restart SIR should start at a reduced dose, which may then gradually be increased to full dose.

If, at any time during the study, an SIR level of <6 ng/mL is obtained, a repeat determination may be performed. Subjects who are unable to tolerate SIR trough levels ≧8 ng/mL due to toxicity may be permanently discontinued from test article, and will start alternative immunosuppression, unless otherwise approved by the principal investigator. Note: This does not apply to SIR trough levels obtained during the initial period of SIR dose titration.

A blood sample for determination of SIR level should be obtained before initiating a reduction in dose. Subjects receiving SIR-based therapy in the absence of calcineurin inhibitors have a higher frequency of hypokalemia and/or hypophosphatemia. In the event of clinically significant electrolyte disturbances, appropriate replacement therapy and further monitoring of electrolytes is recommended. Adjustments may also be indicated to compensate for electrolyte disturbances that may result from diuretic therapy. Report any serious study event, including opportunistic infections, to the WR medical monitor. For subjects whose serum cholesterol or triglyceride concentrations remain >750 mg/dL or >1,000 mg/dL, respectively, despite at least 8 weeks of what, in the judgment of the investigator, is optimal lipid-lowering therapy:

If the trough SIR concentration is ≦8 ng/mL, the patient will discontinue SIR and will start alternative immunosuppression, unless otherwise approved by the principal investigator.

If the corresponding trough SIR concentration is >8 ng/mL, the dose of SIR may be reduced in accordance with table 4 above.

TABLE 6
Myfortic ® or CellCept ® Dose Reductions for Leukopenia

| WBC cells/mm³ | Reduction in current CellCept ® dose |
|---|---|
| ≧3000 | None |
| ≦3000-≧2000 | ↓ by 25% from current dose |
| ≦2000 with ANC ≧ | ↓ by 50% from current dose |
| 1000 cells/mm³ | |
| ANC ≦ 1000 cells/mm³ | Hold until ANC ≧ 1000 cells/mm³ |

Remote capillary glucose monitoring. Glucose monitoring is an important part in patient follow-up after islet transplantation. Real-time communication enables timely therapeutic intervention, which might avoid loss of graft function.

The glucose measuring device is a commercially available capillary glucose meter (LifeScan OneTouch Ultra, manufactured by LifeScan, a division of Johnson and Johnson) which is approved by the FDA for human use as a class 2 medical device. Following the glucose reading, the patient makes the therapeutic decisions regarding glucose control, as with other capillary glucose meters in use.

GLUCOMON® is an automated, wireless blood glucose collection and reporting system accessory to the capillary glucose meter that may be used to send encrypted glucose data through a secure Internet connection for review by the patient and the authorized diabetes care team. The device is manufactured and provided by Diabetech, LP, and is currently used as a non-significant risk investigational device under IRB approved protocols in Phase II and Phase III clinical trials (see ClinicalTrials.gov Identifier: NCT00322478). Diabetech makes no changes to the glucose meter. Further, the GlucoMON does not present data to the patient at the Point of Care when and where decisions about therapy are being made. Following that therapeutic decision made by the patient using the LifeScan OneTouch Ultra glucose meter, the patient may connect the glucose meter to the GlucoMON accessory device to transmit the data.

The GlucoMON then reads the data stored in the glucose meter according to the meter's 510(k) cleared Application Programmer's Interface and within the meter's indications for use. Once the data is copied from the meter, the GlucoMON encrypts and transmits the data to a remote system using a two-way communication protocol to ensure data integrity during communication and storage on the remote system.

Data is delivered through Diabetech's nationwide wireless network to the Diabetech server infrastructure including the hosted patient record. According to our configuration, data is relayed to the Principal Investigator or his designee once per day.

Preparing and delivering the data in real time ensures correct communication of results, enables the health care team to assess effectiveness of glucose control, patient compliance and issues alerts on critical values that necessitate intervention. The use of this system ensures accurate glucose recording and reporting without having to rely solely on the patient.

Diabetech operates as an extension of the provider team and works to ensure patient privacy and security over the patient's data. For the purpose of communicating patient data between Diabetech and the investigators' team, a non-patient identifiable unique identifier may be used to describe the individual, which is not associated with the usual accepted identifiers (such as name, date of birth, identification numbers, as listed in HIPAA) or parts thereof. Diabetech will communicate with the patient including drop shipment of equipment to the patient's home and will perform technical support with the patient to ensure timely and accurate system processing. Diabetech and Baylor University Medical Center or Baylor All Saints Medical Center share a custom patient identifier for the purpose of data transmission, The Principal Investigator and the Diabetech designee hold a protected database linking the custom identifier to actual identifier, upon patient consent. Patient confidentiality and protection of patient data within an authorized care team is always maintained and is described in the Informed Consent document as well.

Before making any changes in treatment, the physician will contact the patient in order to discuss their blood glucose levels and current insulin dosing levels including determining patient compliance with taking the prescribed dose. This will also serve as a verification of the data transmitted electronically.

In addition to the use of the GLUCOMON system, the patient may be required to keep a log of their glucose levels, which includes details which are not captured by the capillary glucose meter, such as diet and exercise level. The GLUCOMON system does not add to the documentation requested from the patient. In addition, the glucose meter itself stores 150 readings which are available to the patient and the medical team during periodic outpatient visits.

For data security purposes, Diabetech and Baylor University Medical Center or Baylor All Saints Medical Center share a custom patient identifier for the purpose of data transmission, which is not associated with the usual accepted identifiers (such as name, date of birth, identification numbers, as listed in HIPAA) or parts thereof. The Principal Investigator and the Diabetech designee hold a protected database linking the custom identifier to actual identifier, upon patient consent.

In order to ensure accurate data protection and storage, Diabetech have multiple security layers in place:

Module Security and Monitoring
1. Private Wireless Network
2. AES Encryption
3. Symmetric Key management
4. Remote Management
5. Embedded Module ID for network Identification

Network and Server Security and Monitoring.
1. Hardware Firewalls
2. Ping Monitoring
3. O/S updates and patches
4. Network Intrusion Detection System (IDS)
5. Service Monitoring
6. System Monitoring
7. Process Monitoring
8. Limited Physical Server Access
9. Hardened Servers with Hardware Firewall
10. Access to servers via public key and secure shell only.
11. Intrusion monitoring and remediation software
12. Distributed Denial of Service (DDOS) monitoring and prevention (Cisco Guard DDOS)
13. State of the art data centers in Dallas, Tex. provide redundant physical server locations 99.9% SLA Guarantee
14. On-site Hands and Eyes
15. 24/7/365 On Site Support
16. Complete redundancy in power, HVAC, fire suppression, network connectivity, and security.
17. Multiple power grids driven by TXU electric, with PowerWare UPS battery backup power and dual diesel generators on-site.
18. HVAC systems are condenser units by Data Aire to provide redundancy in cooling coupled with ten managed backbone providers. Twelve more third party backbone providers are available in the building via cross connect.
19. Fire suppression includes a pre-action dry pipe system including
20. VESDA (Very Early Smoke Detection Apparatus)

Patients are trained on the use of the GLUCOMON device by Diabetech. Since the device relies on a standard OneTouch Ultra meter manufactured by LifeScan, training involves industry norms and only a docking process to trigger the automated data collection along with periodic charging of the portable device. Customer support is also available to the patient if they should have any questions regarding care and maintenance of the technology.

21 CFR Part 11 applies when using a computer system to create, modify, transfer or store an electronic representation of any information or process that is regulated by the Food and Drug Administration (FDA). Diabetech has completed an extensive analysis of its entire system including the GlucoMON device and the procedures utilized in the System Development Life Cycle (SDLC). As 21 CFR Part 11 requires ongoing attention in order to maintain compliance, Diabetech employs personnel responsible for managing the dynamic nature of this regulation.

Diabetech has performed multiple verification and validation tests to ensure data integrity by comparing glucose meter data with data outputs from the system. In addition, we also have internal checks to ensure accurate and complete data collection, communication and storage at each and every step of the process.

In addition, Diabetech may or may not ever commercially sell the current version of the GlucoMON device. In the event that we do decide to seek marketing clearance for this device, Diabetech has taken onto itself the additional burden of compliance under the provisions of the Investigational Device Exemption guidelines of the FDA's Pre Market Approval process. The GlucoMON is deployed as a Non-Significant Risk Investigational Device in some trials. As such, adjustments to therapy should never be made solely on the basis of the data reported by this device.

In essence, the GLUCOMON device and GlucoDYNAMIX reporting system fulfill the role of automating the delivery of the patient's glucose logbook to the care team; a process that usually involves a handwritten log manually transmitted via facsimile.

In summary, Diabetech employs a rigorous software development methodology including data integrity assurances as part of our compliance effort with CFR21Part 11. Diabetech functions under IRB approved protocols which to date have consistently agreed with the Principal Investigator's classification of the GLUCOMON device as presenting ‘Non-Significant Risk’. As such, Diabetech complies through brief and understandable disclosure to the patient within the informed consent and comply with the labeling requirements on the device which describe the investigational nature of the device and that its performance characteristics have not yet been determined.

### Example 1

Seven islet isolations were performed with the ductal injection (DI group) and eight islet isolations were performed without the ductal injection (standard group) using brain-dead donor pancreata. Isolated islets were evaluated based on the Edmonton protocol for transplantation. DI group had significantly higher islet yields (588,566±64,319 IE vs. 354,836±89,649 IE, P<0.01) and viability (97.3±1.2% vs. 92.6±1.2%, P<0.02) compared with the standard group. All seven isolated islet preparations in the DI group (100%), three out of eight isolated islet preparations (38%) met transplantation criteria in the standard group. The islets from the DI group were transplanted into three type 1 diabetic patients and all three patients became insulin independent.

It was found that Ductal Injection (DI) significantly improved quantity of quality of isolated islets resulted in high success rate of clinical islet transplantation. This simple modification will have huge impact on the success of clinical islet transplantation.

Failure to consistently obtain a high quantity and quality of islets is one of the major obstacles for clinical islet transplantation. Even advanced islet centers barely achieved fifty percent of success of clinical islet isolations (1-3). Recently we demonstrated that our modification of Ricordi islet isolation method enabled us to achieve more than 80% of success rate of clinical islet isolation with non-heart-beating donors (NHBDs) (4, 5). This modified islet isolation methods consists of in situ cooling of pancreas after cardiac arrest, ductal preservation with modified Kyoto solution, two-layer pancreas preservation, Ricordi method for pancreas digestion, density adjusted continuous islet purification with iodixanol and Kyoto solution (6). In this study, among those procedures, we introduced the pancreatic ductal injection for brain-dead donors (BDDs) in order to clarify the usefulness of this technique. It revealed that introduction of the pancreatic ductal injection enabled us to achieve seven consecutive success of clinical islet isolation.

MATERIALS AND METHODS. Donor background. Fifteen pancreata from BDDs were procured through either Southwest Transplant Alliance (Dallas, Tex.) or LifeGift (Fort Worth, Tex.) between Apr. 16, 2005 and May 17, 2008. Donor selections were performed based on the Edmonton protocol (7). Donor pancreata were allocated into the ductal injection (DI) group (N=7) or the standard group (N=8).

Pancreata procurement, islet isolation and purification. All pancreata were procured by a transplant surgeon of Baylor Regional Transplantation Institute (Dallas & Fort Worth, Tex.). For the DI group, we removed the duodenum and spleen from the pancreas at the procurement site. This process was performed by Baylor islet team. The pancreas was weighted and a cannula was immediately inserted into the procured pancreas through the main pancreatic duct from the direction of the pancreatic head. Approximately 1 ml/g pancreas of ET-Kyoto solution (Otsuka Pharm Factory Inc., Naruto, Japan) was administered intra-ductally (4-6). For the standard group, the ductal injection process was not performed. All pancreata were preserved by the oxygen static charged two-layer (oxygenated perfluorocarbon/UW solution) method for less than 6 hours (8).

Islet preparations were manipulated according to Good Manufacturing Practice (GMP) at the cell processing facility of Baylor Research Institute in Dallas, Tex. Islet isolation was performed according to the Ricordi method (7, 9). Briefly, after the pancreas was decontaminated, the ducts were perfused in a controlled fashion with a cold enzyme solution. The distended pancreas was then cut into nine pieces and transferred to a Ricordi chamber. The pancreas was digested by repeatedly circulating the enzyme solution through the Ricordi chamber at 37° C. The Phase I period was defined as the time between placement of the pancreas in the Ricordi chamber and the start of collection of the digested pancreas. The Phase II period was defined as the time between the start and the end of the collection.

The islets were purified with a continuous density gradient using Biocoll in a chilled apheresis system (COBE 2991 cell processor, Gambro Laboratories, Denver, Colo.) (7, 9).

Islet evaluation. Islet evaluation was independently judged by two investigators. Islet yield was determined using dithizone staining (Sigma Chemical Co., St. Louis, Mo.) (2 mg/ml) under optical graticule and converted into a standard number of islet equivalents (IE, diameter standardizing to 150 μm) (6, 9). Purity was assessed by comparing the relative quantity of dithizone-stained tissue to unstained exocrine tissue. Islet viability was evaluated using fluorescein diacetate (FDA) and propidium iodide (PI) staining to visualize living and dead cells simultaneously (6, 9).

Islet transplantations into type 1 diabetic patients. Once being islet preparations met the criteria of the Edmonton protocol for transplantation, those isolations were considered successful. Our current criteria for the approval of clinical transplantation are that islets yield more than 4000 IE/kg body weight, purity more than 30%, viability more than 70%, tissue volume less than 10 ml, endotoxin level less than 5 EU/kg body weight and a negative Gram stain based on the Edmonton protocol (7).

Recipient selections were performed based on the Edmonton protocol (7). Patients were sedated and a percutaneous transhepatic approach was used to gain access to the portal vein for all six patients. Once access was confirmed, the Seldinger technique was used to place the Kumpe catheter within the main portal vein. Islets were infused by gravity and using the bag technique (5).

Assessment of transplanted islet function. Islet functioning was assessed in terms of daily serum glucose levels, serum C-peptide, amount of insulin requirement, and HbA1c before and after islet transplantation.

Statistic Analysis. Values for the data collected represent means±SE. Two groups were compared using unpaired t-test. Ratio between two groups was compared Fisher's exact test. P value less than 0.05 is considered significant.

Donor and islet characteristics. Donor-related variables were shown in Table 7. There were no significant differences in the ratio of gender, age, body mass index, peak blood levels of glucose, alanine aminotransferase (ALT) and creatinine.

TABLE 7
Donor-related variables of human pancreas donors

| | | | | Peak Blood Levels of | | |
|---|---|---|---|---|---|---|
| Group | Gender (F/M) | Age (yr) | BMI (kg/m2) | Glucose (mg/dl) | ALT (IU/L) | Crea. (mg/dl) |
| DI | 2/5 | 36.1 ± 4.8 | 31.7 ± 2.8 | 227.6 ± 17.5 | 37.3 ± 8.3 | 1.3 ± 0.2 |
| Standard | 2/6 | 37.5 ± 2.8 | 29.7 ± 1.8 | 221.4 ± 26.1 | 33.1 ± 7.8 | 1.5 ± 0.2 |
| P value | 0.99 | 0.67 | 0.72 | 0.85 | 0.72 | 0.41 |

DI stands for ductal injection, BMI stands for body mass index, ALT stands for alanine aminotransferase, Crea stands for creatinine. Values were expressed as mean ± SE. P value was calculated using Student's t-test except for gender. P value of gender was calculated using Fisher's exact test.

Islet isolation variables were shown in Table 8. There were no significant differences in pancreas weight, cold ischemic time, Phase I period and undigested tissue volume. All pancreata were preserved less than 6 hours. Phase II period was significantly longer in the DI group.

TABLE 8
Islet isolation variables

| Group | Pancreas weight (g) | CIT (min) | Phase I (min) | Phase II (min) | Undigested tissue (g) |
|---|---|---|---|---|---|
| DI | 155.5 ± 16.9 | 157.4 ± 18.3 | 12.7 ± 1.9 | 68.3 ± 3.3 | 18.1 ± 2.9 |
| Standard | 93.3 ± 8.3 | 218.6 ± 20.6 | 15.4 ± 2.2 | 38.7 ± 4.9 | 18.4 ± 2.1 |
| P value | 0.24 | 0.05 | 0.38 | <0.001 | 0.92 |

CIT stands for cold ischemic time. Values were expressed as mean ± SE. P value was calculated using Student's t-test.

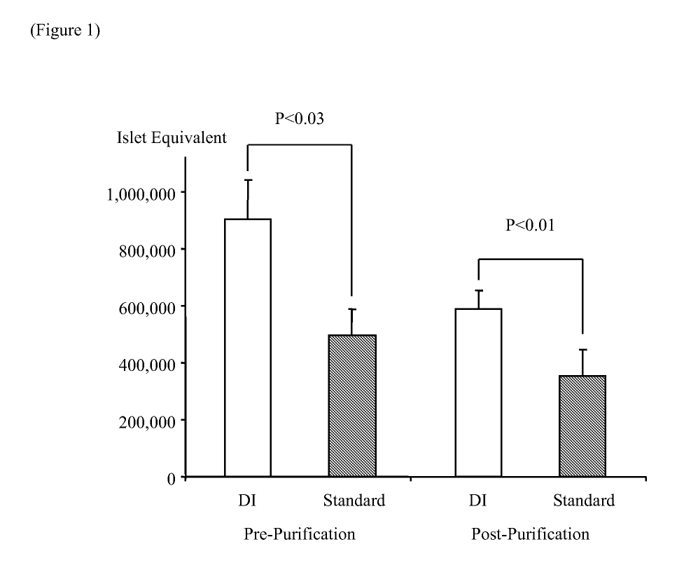

Before islet purification islet yield was significantly higher in DI group (DI vs. Standard; 902,350±139,397 IE vs. 497,457±89,414 IE; P<0.03) (FIG. 1 right). After islet purification, islet yields was also significantly higher in DI group (DI vs. Standard, 588,566±64,319 IE vs. 354,836±89,649 IE; P<0.01) (FIG. 1 left). Islet variables were shown in Table 9. Viability was significantly higher in the DI group. Purity was significantly lower in the DI group.

TABLE 9
Islet variables

| | | | Pellet |
|---|---|---|---|
| Group | Viability (%) | Purity (%) | Size (ml) |
| DI | 97.3 ± 1.2 | 53.3 ± 5.5 | 7.4 ± 1.0 |
| Standard | 92.6 ± 1.2 | 72.9 ± 5.4 | 5.9 ± 1.7 |
| P value | <0.02 | <0.03 | 0.51 |

Values were expressed as mean ± SE. P value was calculated using Student's t-test.

Success of islet isolation. All isolated islets preparations were qualified for transplantation in the DI group (Table 10). Three out of 8 isolated islet preparations were qualified for transplantation in the standard group; the other five had an insufficient islet yield. We attempted to transplant all seven islet preparations in DI group, however, in once case the radiologist could not gain access to portal vein and the preparation was not transplanted. Therefore only six preparations were transplanted into 3 type 1 diabetic patients. Each patient received two islet preparations. In the standard group, two successful preparations were transplanted into two type 1 diabetic patients.

TABLE 10
Qualification for transplantation

| Group | Qualified | Transplanted |
|---|---|---|
| DI | 7/7 (100%) | 6/7 (89%) |
| Standard | 3/8 (37.5%) | 2/8 (25%) |
| P value | <0.03 | <0.05 |

P value was calculated using Fisher's exact test

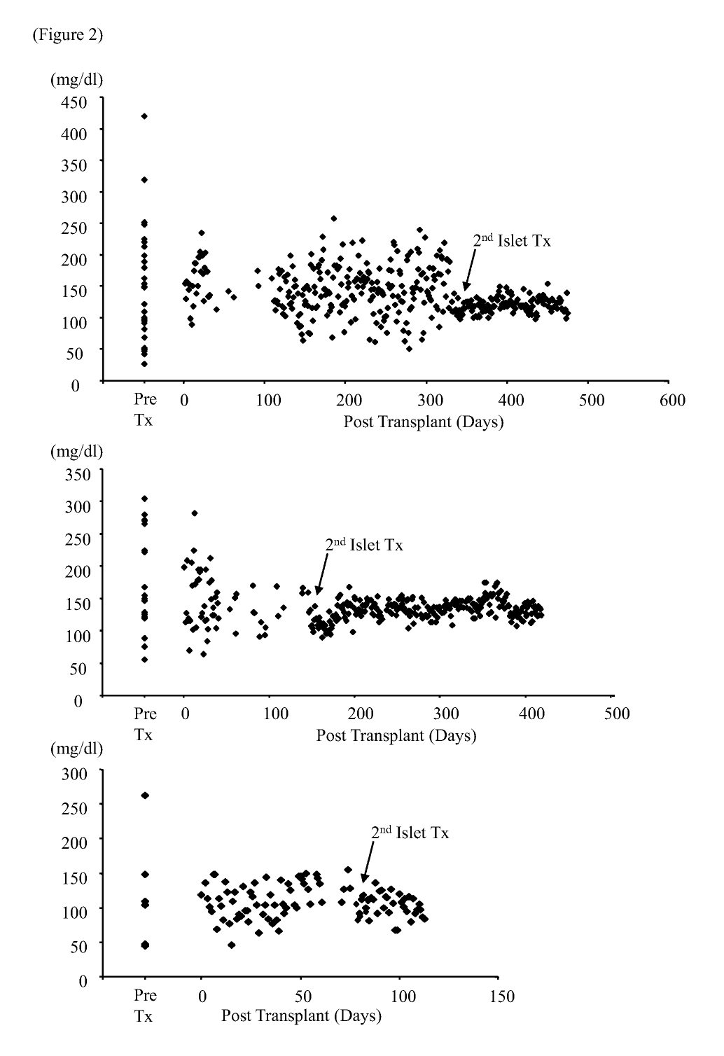

Clinical outcome in the DI group. In the DI group, fasting blood glucose of all three patients improved after single islet transplantation and further improved after the second islet transplantation (FIG. 2). Importantly, after the second islet transplantation, no patients experienced severe hypoglycemia anymore.

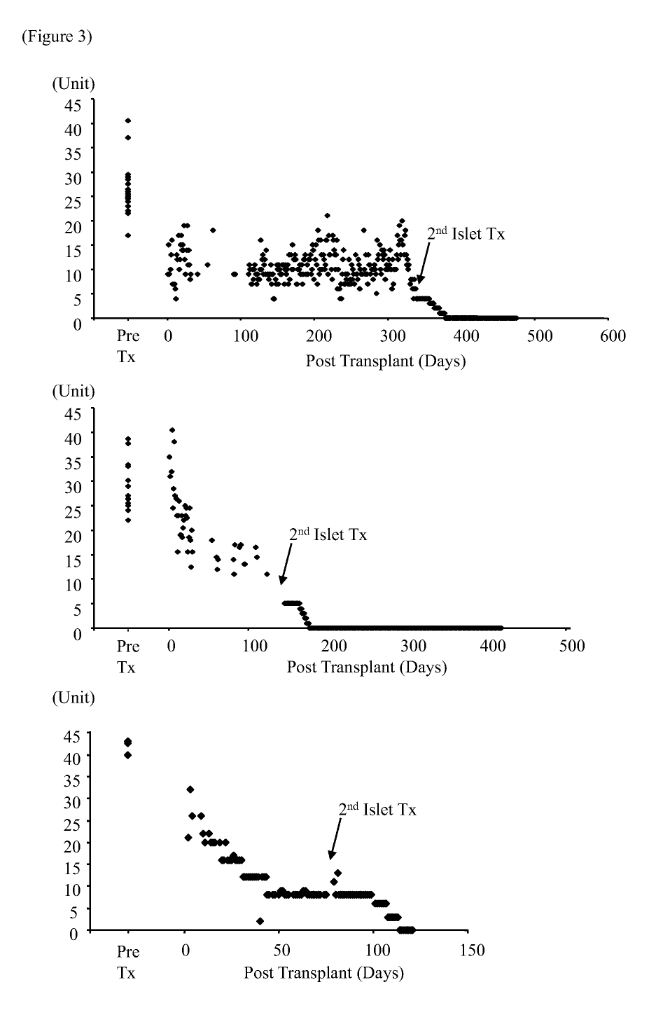

In the DI group, all three patients became insulin independent (FIG. 3). HbA_{1C} before transplantation were 8.3% (first patient), 8.3% (second patient) and 7.4% (third patient) and after transplantation were 6.0%, 5.8% and 5.8% respectively. Fasting C-peptide levels were all undetectable before transplantation. The current fasting C-peptide for the first patient is 2.2 ng/dl, 3.2 ng/dl for the second patient and 2.1 ng/dl for the third patient.

To our knowledge, this is the first study of pancreatic ductal injection at the donor site for clinical islet transplantation using brain-dead donors (BDDs). This simple modification enabled us to have seven consecutive successful clinical islet isolations. Since failure of islet isolation is one of the major issues for clinical islet transplantation (10) because of the loss of donor pancreas, waste of money and efforts, this simple modification is of great value for islet transplantation.

Previously, the present inventors have shown that modification of the Ricordi method including ductal injection improved islet yields using NHBDs (4, 5). For NHBDs, we used ET-Kyoto solution combined with ulinastatin (4, 5); however, ulinastatin was eliminated for this study because ulinastatin is not available in the USA. In addition, usefulness of trypsin inhibition for BDDs is controversial (11, 12). In this study, we confirmed that the ET-Kyoto solution alone was effective for ductal preservation. Usefulness of trypsin inhibition in ductal preservation solution for BDDs is current our research target. Recently we have shown that more than 10% of exocrine tissue suffered apoptic cell death during preservation before islet isolation and the ductal injection of modified Kyoto solution reduced the ratio into less than 2% in porcine model (13). In addition, the ductal injection with both UW solution and modified Kyoto solution improved ATP activity in cellular component in porcine model (13). However, UW solution inhibits collagenase activity (13), and therefore we chose ET-Kyoto solution for human islet isolation.

One of the mechanisms of the ductal injection is protecting both exocrine tissue and islets from apoptotic cell death. Importantly, phase I time was not different between the DI group and the standard group suggested that ductal injection of ET-Kyoto solution did not inhibit the collagenase activity during human pancreas digestion.

Purity was significantly lower in the DI group. It may be that the healthier exocrine tissue survived well during islet isolation process caused lower purity of islet preparations. In addition, significant prolonged phase II time in the DI group suggested that healthier exocrine tissue had less autolysis resulted in prolonged collection period. Since autolyzed exocrine tissues release several digestive enzymes therefore less autolysis could be important to prevent over-digestion of isolated islets. It is reasonable to think that the ductal injection prevents exocrine cell death and therefore leads to avoidance of over-digestion of isolated islets. Concern with low purity is increasing with tissue volume. The tissue volume was higher in the DI group even the difference did not reach statistical significance. However, all islet preparations were adjusted to less than 10 mL and we had no transplant complications related to relatively large tissue volume.

Viability of isolated islets was significantly higher in the DI group. This suggested that the DI also improved the quality of isolated islets.

Sawada et al. demonstrated that the ductal injection of small amount of UW solution protected pancreatic duct in rodent model (14). This is another important mechanism of usefulness of the ductal injection because it is essential to maintain good patency of pancreatic duct for collagenase delivery. Ductal preservation at the procurement site allows us to maintain the patency of the pancreatic duct during preservation and transport; it is therefore possible to use only one cannula for collagenase delivery. The single cannulation technique is better than the usual two cannulations because this technique eliminates cutting pancreas for cannulation. Since the pancreas is not cut, there is excellent pancreas distension and minimization of collagenase leakage.

In this study, approximately 35% of islets were lost during the purification process. Previously, the density was adjusted using ET-Kyoto and iodixanol solution for purification with NHBDs resulted in approximately 80% recovery rate (5). If we were able to achieve the same recovery rate with BDDs, we might be able to obtain more than 700,000 IE from a single donor. Currently, 10,000 IE/kg recipient body weight is the target for insulin independence (7), therefore this high yield would enable us to perform single donor islet transplantation in patients up to 70 kg body weight. Introduction of density adjusted ET-Kyoto and iodixanol solution for purification is currently under investigation at our laboratory.

All three transplanted patients in the DI group became insulin independent, and have improved glycemic control with positive C-peptide. All patients are free from severe hypoglycemia. This clinical outcome shows that the ductal injection is not only useful for obtaining high islet yields but is also contributing for high quality of islets.

In the standard group, one patient received the first islet transplantation and the other patient received the first and second islet transplantation using the islets isolated at the remote center (15) therefore current islet transplantations were their second and third transplantation. Both patients achieved temporal insulin independence after our islet transplantation; however, we were not able to demonstrate that the clinical outcome of the standard group since both patients were received from mixed resources.

In conclusion, the ductal injection of ET-Kyoto solution made us possible to achieve seven consecutive successful clinical islet isolations from BDDs. This simple modification will put huge impact for improving islet isolation and success of clinical islet transplantation.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. LaPorte R E, Matsushima M, Chang Y F: Prevalence and Incidence of Insulin-Dependent Diabetes. In: “Diabetes In America,” 2nd edition. National Diabetes Data Group, National Institutes of Health, National Institute of Diabetes and Digestive and Kidney Diseases, NIH Publication No. 95-1468, 1995, 37-47.
2. Allen R D, Nankivell B J, Hawthorne W J, O'Connell P J, Chapman J R: Pancreas and islet transplantation: an unfinished journey. Transplant Proc 2001; 33(7-8):3485-8.
3. Gruessner R W G, Sutherland D E R, Drangstveit M B, Bland B J, Gruessner A C: Pancreas transplants from living donors: short- and long-term outcome. Transplantation Proc 2001, 33 (1-2): 819-820.
4. Sutherland D E, Gruessner R W, Dunn D L, Matas, A J, Humar A, Kandaswamy R, Mauer S M, Kennedy W R, Goetz F C, Robertson R P, Gruessner A C, Najarian J S: Lessons learned from more than 1,000 pancreas transplants at a single institution. Ann Surg 2001, 233(4): 463-501.
5. Robertson R P, Sutherland D E, Lanz K J: Normoglycemia and preserved insulin secretory reserve in diabetic patients 10-18 years after pancreas transplantation. Diabetes 1999, 48(9): 1737-1740.
6. Fiorina P, La Rocca E, Astorri E, Lucignani G, Rossetti C, Fazio F, Giudici D, di Carlo V, Cristallo M, Pozza G, Secchi A: Reversal of left ventricular diastolic dysfunction after kidney-pancreas transplantation in type 1 diabetic uremic patients. Diabetes Care 2000, 23(12): 1804-1810.
7. Allen R D, Al-Harbi I S, Morris J G, Clouston P D, O'Connell P J, Chapman J R, Nankivell B J: Diabetic neuropathy after pancreas transplantation: determinants of recovery. Transplantation 1997, 63(6): 830-838.
8. Chow V C C, Pai R P, Chapman J R, O'Connell P J, Allen R D M, Mitchell P, Nankivell B J: Diabetic retinopathy after combined kidney-pancreas transplantation. Clinical Transplantation 1999, 13(4): 356-362.
9. Sollinger H W, Odorico J S, Knechtle S J, D'Alessandro A M, Kalayoglu M, Pirsch J D: Experience with 500 simultaneous pancreas-kidney transplants. Ann Surg 1998, 228(3):284-96.
10. Reddy K S, Stratta R J, Shokouh-Amiri M H, Alloway R, Egidi M F, Gaber A O: Surgical complications after pancreas transplantation with portal-enteric drainage. J Am Coll Surg 1999; 189(3): 305-313.
11. Gruessner A C, Sutherland D E: Pancreas transplant outcomes for United States (US) cases reported to the United Network for Organ Sharing (UNOS) and non-US cases reported to the International Pancreas Transplant Registry (IPTR) as of October, 2000. Clinical Transplants 2000, (1): 45-72.
12. 2001 Annual Report of the U.S. Organ Procurement and Transplantation Network and the Scientific Registry for Transplant Recipients: Transplant Data 1991-2000. Department of Health and Human Services, Health Resources and Services Administration, Office of Special Programs, Division of Transplantation, Rockville, Md.; United Network for Organ Sharing, Richmond, Va.; University Renal Research and Education Association, Ann Arbor, Mich.
13. Linetsky E, Bottino R, Lehmann R, Alejandro R, Inverardi L, Ricordi C: Improved human islet isolation using a new enzyme blend, liberase. Diabetes 1997, 46(7):1120-1123.
14. Lakey J R T, Warnock G L, Shapiro A M J, Korbutt G S, Ao Z, Kneteman N M, Rajotte R V: Intraductal collagenase delivery into the human pancreas using syringe loading or controlled perfusion. Cell Transplant 1999, 8(3):285-292.
15. Ricordi C, Lacy P E, Scharp D W: Automated islet isolation from human pancreas. Diabetes 1989, 38 (Suppl. 1): 140-142.
16. Shapiro A M J, Lakey J R T, Ryan E A, Korbutt G S, Toth E L, Warnock G L, Kneteman N N, Rajotte R V: Islet transplantation in seven patients with Type 1 diabetes mellitus using a glucocorticoid-free immunosuppressive regimen. N Engl J Med 343:230-238, 2000.
17. Rutzky L P, Bilinski S, Kloc M, Phan T, Zhang H, Katz S M, Stepkowski S M: Microgravity culture condition reduces immunogenicity and improves function of pancreatic islets. Transplantation 2002 Jul. 15, 74(1): 13-21.
18. Gaber A O, Fraga D W, Callicutt C S, Gerling I C, Sabek O M, Kotb M Y: Improved in vivo pancreatic islet function after prolonged in vitro islet culture. Transplantation 2001, 72(11): 1730-1736.
19. Matarazzo M, Giardina M G, Guardasole V, Davalli A M, Horton E S, Weir G C, Sacca L, Napoli R: Islet transplantation under the kidney capsule corrects the defects in glycogen metabolism in both liver and muscle of streptozocin-diabetic rats. Cell Transplant 2002, 11(2): 103-112.
20. Hirshberg B, Montgomery S, Wysoki M G, Xu H, Tadaki D, Lee J, Hines K, Gaglia J, Patterson N, Leconte J, Hale D, Chang R, Kirk A D, Harlan D M: Pancreatic islet transplantation using the nonhuman primate (rhesus) model predicts that the portal vein is superior to the celiac artery as the islet infusion site. Diabetes 2002, 51(7): 2135-2140.
21. Levy M M, Ketchum R J, Tomaszewski J E, Naji A, Barker C F, Brayman K L: Intrathymic islet transplantation in the canine: I. Histological and functional evidence of autologous intrathymic islet engraftment and survival in pancreatectomized recipients. Transplantation 2002, 73(6): 842-852.
22. Drachenberg C B, Klassen D K, Weir M R, Wiland A, Fink J C, Bartlett S T, Cangro C B, Blahut S, Papadimitriou J C: Islet cell damage associated with tacrolimus and cyclosporine: morphological features in pancreas allograft biopsies and clinical correlation. Transplantation 1999, 68(3): 396-402.
23. Ricordi C, Strom, T B: Clinical islet transplantation: Advances and immunological challenges. Nat Rev Immunol. 2004 April; 4(4):259-68.
24 Ryan E A, Paty B W, Senior P A, Bigam D, Alfadhli E, Kneteman N M, Lakey J R T, Shapiro A M J. Five-year follow-up after clinical islet transplantation. Diabetes 2005 54:2060-2069
25. Froud T, Ricordi C, Baidal D A, Hafiz M H, Ponte G, Cure P, Pileggi A, Poggioli R, Ichii H, Khan A, Ferreiraa J V, Pugliese A, Esquenazi V V, Kenyon N S, Alejandro R. Islet transplantation in type 1 diabetes mellitus using cultured islets and steriod-free immunosuppression: Miami experience. Am J Transplantation 2005 5(8):2037-2046
26. Hering B J, Kandaswamy R, Ansite J D, Eckman P M, Nakano M, Sawada T, Matsumoto I, Ihm S-H, Zhang H-J, Parkey J, Hunter D W, Sutherland D E R. Single-donor, marginal-dose islet transplantation in patients with type 1 diabetes JAMA 2005 293(7):830-835
27. Markmann J F, Rosen M, Sigelman E S, Soulen M C, Deng S, Barker C F, Naji A. Magnetic resonance-defined periportal steatosis following intraportal islet transplantation: a functional footprint of islet graft survival? Diabetes 2003 52(7):1591-1594
28. Ryan E A, Lakey J R, Rajotte, R V, Korbutt G S, Kin T, Imes S, Rabinovitch A, Elliott J F, Bigam D: Kneteman N M, Warnock G L, Larsen I, Shapiro A M: Clinical outcomes and insulin secretion after islet transplantation with the Edmonton protocol. Diabetes 2001, 50(4): 710-719.
29 Owen, R J T, Ryan, E A, O'Kelly, K, Lakey, J R T, McCarthy, M C, Paty, B W, Bigam, D L, Kneteman, N M, Korbutt, G S, Rajotte, R V, Shapiro, A M J: Percutaneous transhepatic pancreatic islet cell transplantation in type 1 diabetes mellitus: Radiologic aspects. Radiology 2003, 229: 165-170.
30. Matsumoto S, Qualley S A, Goel S, Hagman D K, Sweet I R, Poitout V, Strong D M, Robertson R P, Reems J A: Effect of the two-layer (University of Wisconsin solution-perfluorochemical plus O2) method of pancreas preservation on human islet isolation, as assessed by the Edmonton Isolation Protocol. Transplantation 2002, 74(10): 1414-1419
31. Rastellini C, Braun M, Cicalese L, Benedetti E: Construction of an optimal facility for clinical pancreatic islet isolation. Transplant Proc 2001, 33(7-8): 3524.
32. Matsumoto S, Okitsu T, Iwanaga Y, Noguchi H, Nagata H, Yonekawa Y, Yamada Y, Fukuda K, Shibata T, Kasai Y, Maekawa T, Wada H, Nakamura T, Tanaka K. Successful islet transplantation from nonheartbeating donor pancreata using modified Ricordi islet isolation method. Transplantation 2006 82(4):460-5.
33. Ryan E A, Shandro T, Green K, Paty B W, Senior P A, Bigam D, Shapiro A M, Vantyghem M C. Assessment of the severity of hypoglycemia and glycemic lability in type 1 diabetic subjects undergoing islet transplantation. Diabetes. 2004 April; 53(4):955-62.
34. Levy M F, Jennings L, Abouljoud M S, Mulligan D C, Goldstein R M, Husberg B S, Gonwa T A, Klintmalm G B. Quality of life improvements at one, two, and five years after liver transplantation Transplantation 1995 59(4):515-518.

### References Example 1

Kin T, Mirbolooki M, Salehi P, et al. Islet isolation and transplantation outcomes of pancreas preserved with University of Wisconsin solution versus two-layer method using preoxygenated perfluorocarbon. Transplantation 2006; 82:1286.
Matsumoto I, Sawada T, Nakano et al. Improvement in islet yield from obese donors for human islet transplants. Transplantation 2004; 78:880.
Ichii H, Pileggi A, Molano R D, et al. Rescue purification maximizes the use of human islet preparations for transplantation. Am J Transplant 2005; 5: 21.
Matsumoto S, Tanaka K. Pancreatic islet cell transplantation using non-heart-beating donors (NHBDs). J Hepatobiliary Pancreat Surg 2005; 12: 227.
Matsumoto S, Okitsu T, Iwanaga Y, et al. Successful islet transplantation from nonheartbeating donor pancreata using modified Ricordi islet isolation method. Transplantation 2006; 82:460.
Matsumoto S, Noguchi H, Naziruddin B, et al. Improvement of pancreatic islet isolation for transplantation. Baylor University Medical Center Proceedings 2007; 20: 357.
Shapiro A M, Lakey J R, Ryan E A, et al. Islet transplantation in seven patients with type 1 diabetes mellitus using a glucocorticoid-free immunosuppressive regimen. N Engl J Med 2000; 343:230.
Matsumoto S, Rigley T H, Qualley S A, et al. Efficacy of the oxygen-charged static two-layer method for short-term pancreas preservation and islet isolation from nonhuman primate and human pancreata. Cell Transplant 2002; 11: 769.
Matsumoto S, Qualley S, Goel S, et al. Effect of the two-layer (University of Wisconsin solution-perfluorochemical plus O2) method of pancreas preservation on human islet isolation, as assessed by the Edmonton Isolation Protocol. Transplantation 2002; 74: 1414.
Markmann J F, Kaufman D B, Ricordi C, Schwab P M, Stock P G. Financial issues constraining the use of pancreata recovered for islet transplantation: A white paper. Am J Transplant 2008; 8: 1.
Rose N L, Palcic M M, Helms L M, Lakey J R. Evaluation of Pefabloc as a serine protease inhibitor during human-islet isolation. Transplantation 2003; 75:462.
Matsumoto S, Rigley T, Reems J, et al. Improved islet yields from macaca nemestrina and marginal human pancreata after two-layer method preservation and endogenous trypsin inhibition. Am J Transplant 2003; 3: 53.
Noguchi H, Ueda M, Hayashi S et al. Ductal injection of preservation solution increases islet yields in islet isolation and improves islet graft function. Cell Transplant 2008; 17: 69.
Sawada T, Matsumoto I, Nakano M, Kirchhof N, Sutherland D E, Hering B J. Improved islet yield and function with ductal injection of University of Wisconsin solution before pancreas preservation. Transplantation 2003; 75: 1965.
Ichii H, Sakuma Y, Pileggi A, et al. Shipment of human islets for transplantation. Am J Transplant 2007; 7: 1010.

What is claimed is:

1. A method of preparing a transplantable islet preparation, the method comprising the steps of:
harvesting the pancreas of a donor;
injecting the pancreatic ducts with ET-Kyoto solution or equivalent thereto;
isolating pancreatic β-islet cells; and
treating the patient with a human interleukin-1 antagonist at the time of islet transplant.

2. The method of claim 1, wherein the step of isolating the pancreatic β-islet cells is performed using a collagenase.

3. The method of claim 2, wherein the collagenase comprises a human collagenase.

4. The method of claim 1, wherein the islets are processed in ET-Kyoto solution after their extraction from the pancreas.

5. The method of claim 1, wherein the step of isolating the pancreatic β-islet cells is conducted in the presence of a trypsin inhibitor.

6. The method of claim 1, wherein the human interleukin-1 antagonist is selected from: one or more modifiers of interleukin-1 beta (IL-1β) gene transcription; one or more modifiers of IL-1β gene translation; one or more siRNAs that target the expression of IL-1β; one or more IL-1β receptors blockers; one or more interleukin-1 receptor antagonist proteins; one or more interleukin-1 receptor antagonist peptides; one or more active agents that modify the release of IL-1β; one or more antibodies that neutralize IL-1β; one or more antibodies that blocks an IL-1β receptor; one or more recombinant, naturally occurring IL-1 receptor antagonists; one or more anion transport inhibitors, lipoxins and alpha-tocopherol that inhibit the release of IL-1β; one or more opioids that inhibits a proteolytic enzyme that converts the inactive IL-1β precursor to its mature, active form; one or more antibodies that neutralizes the biological function of IL-1β, mixtures and combinations thereof.

7. The method of claim 1, further comprising providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.

8. A method of preparing a transplantable islet preparation, the method comprising the steps of:
harvesting the pancreas of a donor;
injecting the pancreatic ducts with ET-Kyoto solution or equivalent thereto;
isolating pancreatic β-islet cells from the harvested pancreas in the presence of a trypsin inhibitor; and
treating the patient with a human interleukin-1 antagonist at the time of islet transplant.

9. The method of claim 8, wherein the trypsin inhibitor is selected from a serum α-1 antitrypsin, a lima bean trypsin inhibitor, a Kunitz inhibitor, a ovomucoid inhibitor or a soybean inhibitor.

10. The method of claim 8, wherein the pancreatic islets are processed in ET-Kyoto solution after their extraction from the pancreas.

11. The method of claim 8, wherein the pancreatic islets are processed with a collagenase.

12. The method of claim 11, wherein the collagenase comprises a human collagenase.

13. The method of claim 8, wherein the human interleukin-1 antagonist is selected from: one or more modifiers of interleukin-1 beta (IL-1β) gene transcription; one or more modifiers of IL-1β gene translation; one or more siRNAs that target the expression of IL-1β; one or more IL-1β receptors blockers; one or more interleukin-1 receptor antagonist proteins; one or more interleukin-1 receptor antagonist peptides; one or more active agents that modify the release of IL-1β; one or more antibodies that neutralize IL-1β; one or more antibodies that blocks an IL-1β receptor; one or more recombinant, naturally occurring IL-1 receptor antagonists; one or more anion transport inhibitors, lipoxins and alpha-tocopherol that inhibit the release of IL-1β; one or more opioids that inhibits a proteolytic enzyme that converts the inactive IL-1β precursor to its mature, active form; one or more antibodies that neutralizes the biological function of IL-1β, mixtures and combinations thereof.

14. The method of claim 8, further comprising providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.

15. A method of preparing a transplantable islet preparation, the method comprising the steps of:
harvesting the pancreas of a donor;
isolating pancreatic β-islet cells isolating pancreatic β-islet cells from the harvested pancreas in the presence of a trypsin inhibitor; and
treating the patient with a human interleukin-1 antagonist and a Tumor Necrosis Factor antagonist at the time of islet transplant.

16. The method of claim 15, wherein the extraction is performed using a suitable collagenase in ET-Kyoto solution.

17. The method of claim 15, wherein the islets are processed in ET-Kyoto solution after their extraction from the pancreas.

18. The method of claim 15, wherein the trypsin inhibitor is selected from a serum α-1 antitrypsin, a lima bean trypsin inhibitor, a Kunitz inhibitor, a ovomucoid inhibitor or a soybean inhibitor.

19. The method of claim 15, wherein the collagenase comprises a human collagenase.

20. The method of claim 15, further comprising providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.
